(19) United States
(12) Patent Application Publication
(10) Pub. No.: US 2019/0002546 A1
(43) Pub. Date: Jan. 3, 2019

(54) METHODS AND COMPOSITIONS FOR BINDING VEGF

(71) Applicant: Zhuhai Tairuishang Biopharm Ltd., Guangdong (CN)

(72) Inventors: Ronghao Li, Foster City, CA (US); John Jun Wu, Thousand Oaks, CA (US)

(21) Appl. No.: 15/985,541

(22) Filed: May 21, 2018

Related U.S. Application Data
(63) Continuation of application No. PCT/CN2016/106399, filed on Nov. 18, 2016.
(60) Provisional application No. 62/257,673, filed on Nov. 19, 2015.

(51) Int. Cl. C07K 16/22; C12N 15/62; A61P 35/00; A61P 27/00

(52) U.S. Cl. CPC C07K 16/22; C12N 15/62; A61P 35/00; A61P 27/00; A61K 9/0019; C07K 2319/01; C07K 2317/92; C07K 2317/76; C07K 2319/74

(57) ABSTRACT

The present invention relates to fusion polypeptide compositions comprising immunoglobulin-type-2 binding domains of Vascular Endothelial Growth Factor Receptor, isolated nucleic acids encoding the compositions and vectors and host cells containing the same, and methods of using such compositions in treatment of diseases, disorders, and conditions.

### CROSS-REFERENCE

This application is a continuation application of International Application No. PCT/CN2016/106399, filed on Nov. 18, 2016, which claims the benefit of U.S. Provisional Application No. 62/257,673, filed on Nov. 19, 2015, which disclosure is hereby incorporated by reference in its entirety.

### BACKGROUND OF THE INVENTION

Vascular Growth Endothelial Factor (VEGF) is a polypeptide ligand that stimulates vasculogenesis and angiogenesis. Overexpression of VEGF can play a role in diseases such as cancer and diseases of the retina. VEGF signaling is mediated by several VEGF receptors that contain an extracellular domain capable of binding VEGF, and a tyrosine kinase domain responsible for initiating a signaling cascade. VEGF inhibitors can halt or slow the progression of diseases mediated by neovascularization by preventing VEGF receptor signaling. Improved inhibition of VEGF receptor signaling can result in better patient outcomes, for example by further slowing the progressive loss of sight due to wet macular degeneration.

### SUMMARY OF THE INVENTION

There exists a considerable need for alternative therapeutics that inhibit VEGF signalling. The present invention addresses this need and provides additional advantages. In one aspect, the present invention provides for a fusion polypeptide comprising two or more VEGF receptor immunoglobulin-like-type 2 domains fused with a dimerization polypeptide. In some embodiments provided herein, at least one of the two or more VEGF receptor immunoglobulin-like-type 2 domains is fused to an N-terminus of the dimerization polypeptide and at least another of the two or more VEGF receptor immunoglobulin-like-type 2 is fused to a C-terminus of the dimerization polypeptide. In some embodiments provided herein, the dimerization polypeptide is a fragment crystallizable (Fc) domain. In some embodiments provided herein, the dimerization domain comprises a first cysteine residue capable of forming a disulfide bond to a second cysteine residue.

In some embodiments provided herein, the fusion polypeptide further comprises at least one hinge region between the dimerization polypeptide and the two or more VEGF receptor immunoglobulin-like-type 2 domains. In some embodiments provided herein, the two or more VEGF receptor immunoglobulin-like-type 2 domains are each at least 80% identical to a human VEGF receptor immunoglobulin-like-type 2 domain selected from the group consisting of SEQ ID NOs 4-6. In some embodiments provided herein, the two or more VEGF receptor immunoglobulin-like-type 2 domains are each at least 90% identical to a human VEGF receptor immunoglobulin-like-type 2 domain selected from the group consisting of SEQ ID NOs 4-6. In some embodiments provided herein, the two or more VEGF receptor immunoglobulin-like-type 2 domains are each at least 95% identical to a human VEGF receptor immunoglobulin-like-type 2 domain selected from the group consisting of SEQ ID NOs 4-6. In some embodiments provided herein, the two or more VEGF receptor immunoglobulin-like-type 2 domains are each a human VEGF receptor immunoglobulin-like-type 2 domain selected from the group consisting of SEQ ID NOs 4-6.

In some embodiments provided herein, the two or more VEGF receptor immunoglobulin-like-type 2 domains are each SEQ ID NO: 4. In some embodiments provided herein, the two or more VEGF receptor immunoglobulin-like-type 2 domains are each SEQ ID NO: 5. In some embodiments provided herein, the two or more VEGF receptor immunoglobulin-like-type 2 domains are each SEQ ID NO: 6. In some embodiments provided herein, the two or more VEGF receptor immunoglobulin-like-type 2 domains are not identical. In some embodiments provided herein, the two or more VEGF receptor immunoglobulin-like-type 2 domains are at least two distinct human VEGF receptor immunoglobulin-like-type 2 domains. In some embodiments provided herein, the at least two distinct human VEGF receptor immunoglobulin-like-type 2 domains are selected from the group consisting of SEQ ID NOs: 4-6.

In some embodiments provided herein, the fusion polypeptide substantially lacks a VEGF receptor immunoglobulin-like-type 3 domain selected from the group consisting of SEQ ID NOs 1-3. In some embodiments provided herein, the fusion polypeptide comprises no more than 60 amino acids of a VEGF receptor immunoglobulin-like-type 3 domain selected from the group consisting of SEQ ID NOs 1-3. In some embodiments provided herein, the VEGF receptor immunoglobulin-like-type 3 domain is at least 80% identical to a human VEGF receptor immunoglobulin-like-type 3 domain selected from the group consisting of SEQ ID NOs 1-3. In some embodiments provided herein, the VEGF receptor immunoglobulin-like-type 3 domain is at least 90% identical to a human VEGF receptor immunoglobulin-like-type 3 domain selected from the group consisting of SEQ ID NOs 1-3. In some embodiments provided herein, the VEGF receptor immunoglobulin-like-type 3 domain is at least 95% identical to a human VEGF receptor immunoglobulin-like-type 3 domain selected from the group consisting of SEQ ID NOs 1-3. In some embodiments provided herein, the human VEGF receptor immunoglobulin-like-type 3 domain is SEQ ID NO: 1. In some embodiments provided herein, the human VEGF receptor immunoglobulin-like-type 3 domain is SEQ ID NO: 2. In some embodiments provided herein, the human VEGF receptor immunoglobulin-like-type 3 domain is SEQ ID NO: 3. In some embodiments provided herein, the fusion polypeptide does not comprise SEQ ID NO: 8.

In some embodiments provided herein, a homodimer of the fusion polypeptide exhibits high-affinity binding to VEGF. In some embodiments provided herein, the homodimer binds to VEGF with a higher affinity than the polypeptide shown in SEQ ID NO: 9. In some embodiments provided herein, the homodimer binds to VEGF with a higher affinity than a human VEGF receptor.

In one aspect, the present invention provides a fusion polypeptide comprising two or more vascular endothelial growth factor (VEGF) receptor immunoglobulin-like-type 2 domains fused to a dimerization polypeptide, wherein a first homodimer of the fusion polypeptide binds VEGF with a higher affinity than a second homodimer of aflibercept (SEQ ID NO: 9) when measured by: incubating the first homodimer and the second homodimer separately with immobilized VEGF to produce bound complexes; washing the bound complexes to remove non-specific binding; and performing enzyme-linked immunosorbent assay (ELISA) to assess an amount of the bound complexes after step (b).

In one aspect, the present invention provides an isolated polynucleotide molecule encoding the fusion polypeptide of any of the foregoing embodiments.

In one aspect, the present invention provides an isolated polynucleotide molecule encoding a fusion polypeptide that forms a homodimer capable of binding vascular epithelial growth factor (VEGF), wherein the polynucleotide comprises SEQ ID NO: 7.

In one aspect, the present invention provides a vector comprising a sequence of the isolated polynucleotide molecule of any of the foregoing aspects and embodiments.

In one aspect, the invention provides a cell comprising any of the foregoing vectors or polynucleotides. In some embodiments provided herein, the cell is a mammalian cell. In some embodiments provided herein, the cell is a bacterial cell. In some embodiments provided herein, the cell is a fungal cell. In some embodiments provided herein, the cell is an insect cell.

In one aspect, the invention provides a method for inhibiting angiogenesis in a subject in need thereof comprising: administering to the subject in need thereof a therapeutically effective amount of a homodimer of any of the foregoing fusion polypeptides. In some embodiments provided herein, the administering is effected by a local administration or a systemic administration to the subject. In some embodiments provided herein, the administration is to an eye of the subject. In some embodiments provided herein, the administration is to a tumor tissue of the subject. In some embodiments provided herein, the administration is intravenous injection. In some embodiments provided herein, the administration is intraperitoneal injection. In some embodiments provided herein, the administration is intravitreal injection.

In some embodiments provided herein, in any of the foregoing methods, the angiogenesis is a manifestation of a condition selected from the group consisting of age-related macular degeneration, diabetic retinopathy, choroidal neovascularization, cystoid macular edema, diabetic macular edema, retinal vascular occlusion, corneal neovascularization, corneal transplantation, neovascular glaucoma, pterygium chronic conjunctivitis, angiogenesis related therapy failure such as laser coagulation, and surgical retinal transplantation. In some embodiments provided herein, the condition is AMD. In some embodiments provided herein, the condition is diabetic retinopathy.

In some embodiments provided herein, the administering results in one or more improved symptoms of the condition, wherein the symptoms are selected from the group consisting of a decrease in mean choroidal neovascularization (CNV) leakage, improved mean visual acuity, a reduction in mean foveal retinal thickness, a reduction in mean macular size, and a reduction in mean lesion size. In some embodiments provided herein, the one or more improved symptoms of the condition remains improved for at least 1 month following the administration.

In some embodiments provided herein, the homodimer is administered by intravitreal injection at an amount from about 1 mg to about 3 mg. In some embodiments provided herein, the homodimer is administered by intravitreal injection of an amount of about 2 mg. In some embodiments provided herein, the angiogenesis is a manifestation of a tumor. In some embodiments provided herein, the fusion polypeptide is administered by intravenous injection. In some embodiments provided herein, the fusion polypeptide is administered by an intravenous injection comprising an amount of from about 0.1 to about 30 mg/kg, or from about 1 to about 8 mg/kg.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the structure of an exemplary VEGF-binding dimer of a polypeptide described herein.

FIGS. 2A-C illustrate dot blots of CHO cells expressing fusion polypeptide-1 and Western blot analysis of the purified protein.

FIGS. 3A-D illustrate the neutralization of VEGF by fusion polypeptide-1 in vitro and in culture.

FIG. 4 illustrates a comparison of in vitro VEGF neutralization by Aflibercept (Eylea), Conbercept, and fusion polypeptide-1.

FIGS. 5A-D illustrate the pharmacokinetics of fusion polypeptide-1 by demonstrating its retention and effectiveness after injection into mice.

FIGS. 6A-B illustrate the neutralization of VEGF using sera extracted from mice injected with fusion polypeptide-1.

FIGS. 7A-I illustrate the in vivo neutralization of laser injury-induced neovascularization by fusion polypeptide-1.

### DETAILED DESCRIPTION OF THE INVENTION

The systems and methods of this disclosure as described herein may employ, unless otherwise indicated, conventional techniques and descriptions of molecular biology (including recombinant techniques), cell biology, biochemistry, microarray and sequencing technology, which are within the skill of those who practice in the art. Such conventional techniques include polymer array synthesis, hybridization and ligation of oligonucleotides, sequencing of oligonucleotides, and detection of hybridization using a label. Specific illustrations of suitable techniques can be had by reference to the examples herein. However, equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, et al., Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Stryer, L., Biochemistry (4th Ed.) W.H. Freeman, N.Y. (1995); Gait, “Oligonucleotide Synthesis: A Practical Approach” IRL Press, London (1984); Nelson and Cox, Lehninger, Principles of Biochemistry, 3^{rd} Ed., W.H. Freeman Pub., New York (2000); and Berg et al., Biochemistry, 5^{th} Ed., W.H. Freeman Pub., New York (2002), all of which are herein incorporated by reference in their entirety for all purposes. Before the present compositions, research tools and systems and methods are described, it is to be understood that this disclosure is not limited to the specific systems and methods, compositions, targets and uses described, as such may, of course, vary. It is also to be understood that the terminology used herein is for the purpose of describing particular aspects only and is not intended to limit the scope of the present disclosure, which will be limited only by appended claims.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms “polynucleotide,” “nucleic acid,” and “oligonucleotide” are used interchangeably. As used herein, they generally refer to a polymeric form of nucleotides of any length, either deoxyribonucleotides or ribonucleotides, or analogs thereof. Polynucleotides may have any three dimensional structure, and may perform any function, known or unknown. Non-limiting examples of polynucleotides are coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, cDNA, recombinant polynucleotides, branched polynucleotides, plasmids, vectors, isolated DNA of any sequence, isolated RNA of any sequence, nucleic acid probes, adapters, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “polypeptide”, “peptide” and “protein” are used interchangeably herein to refer to polymers of amino acids of any length. The polymer may be linear or branched, it may comprise modified amino acids, and it may be interrupted by non amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

A “control” is an alternative subject or sample used in an experiment for comparison purpose.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells, and their progeny of a biological entity obtained in vivo or cultured in vitro are also encompassed.

The terms “determining”, “measuring”, “evaluating”, “assessing,” “assaying,” and “analyzing” can be used interchangeably herein to refer to any form of measurement, and include determining if an element is present or not (for example, detection). These terms can include both quantitative and/or qualitative determinations. Assessing may be relative or absolute. “Detecting the presence of” can include determining the amount of something present, as well as determining whether it is present or absent.

In general, “sequence identity” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (i.e., nucleotides or amino acids), divided by the total number of symbols in the shorter of the two sequences. The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 80% to 100% and integer values therebetween. In general, an exact match indicates 100% identity over the length of the shortest of the sequences being compared (or over the length of both sequences, if identical).

A “chimeric” or “fusion” polypeptide contains at least one polypeptide comprising regions in a different position in the sequence than that which occurs in nature. The regions may normally exist in separate proteins and are brought together in the fusion polypeptide; or they may normally exist in the same protein but are placed in a new arrangement in the fusion polypeptide. A chimeric or fusion polypeptide protein may be created, for example, by chemical synthesis, or by creating and translating a polynucleotide in which the peptide regions are encoded in the desired relationship.

“Immunoglobulin-like domain” or “Ig-like domain” or “ligand-binding domain” refers to independent and distinct domains that are found in the extracellular ligand-binding region of cytokine receptors and it is specifically intended that the term encompass not only the complete wild-type domain, but also insertion, deletion and substitution variants thereof that retain at least a portion of the binding affinity of the wild-type domain. It will be readily apparent to those of ordinary skill in the art that numerous variants of the domains or combinations of the domains of the cytokine binding proteins can be obtained which will retain substantially the same functional characteristics as the wild type domain.

The phrase “ophthalmically acceptable” with respect to a formulation, composition or ingredient herein means having no persistent effect that is substantially detrimental to the treated eye or the functioning thereof, or on the general health of the subject being treated. It will be recognized that transient effects such as minor irritation or a “stinging” sensation are common with topical ophthalmic administration of drugs and the existence of such transient effects is not inconsistent with the formulation, composition or ingredient in question being “ophthalmically acceptable” as herein defined. However, preferred formulations, compositions and ingredients are those that cause no substantial detrimental effect, even of a transient nature.

In one aspect, the present disclosure provides compositions comprising fusion polypeptides that include VEGF receptor immunoglobulin-like-type 2 domains fused with a dimerization polypeptide.

In another aspect, the present disclosure provides compositions comprising the fusion polypeptide comprising two or more vascular endothelial growth factor (VEGF) receptor immunoglobulin-like-type-2 domains fused to a dimerization polypeptide, wherein a first homodimer of the fusion polypeptide binds VEGF with a higher affinity than a second homodimer of aflibercept (SEQ ID NO: 9) when measured by: (a) incubating the first homodimer and the second homodimer separately with immobilized VEGF to produce bound complexes; (b) washing the bound complexes to remove non-specific binding; and (c) performing enzyme-linked immunosorbent assay (ELISA) to assess an amount of the bound complexes after step (b).

VEGF receptor immunoglobulin-like-type-2 domains are generally found in all members of the VEGF receptor family. In some cases, VEGF receptors can be membrane bound. In some cases, VEGF receptors can be soluble. In some cases, a VEGF receptor is one of three subtypes. For example, the first subtype can be referred to as VEGFR-1. Non-limiting examples of the first subtype are mouse and human Flt1. The second subtype can be referred to as VEGFR-2. Non-limiting examples of the second subtype are human and mouse Kdr, which can be referred to as Flk1 or cluster of differentiation 309 (CD309). The third subtype can be referred to as VEGFR-3. Non-limiting examples of the second subtype are human and mouse Flt-4.

The dimerization polypeptide utilized in the fusion polypeptide can be a fragment crystallizable (Fc) region of an antibody. In some cases, the dimerization polypeptide includes a hinge region. The dimerization polypeptide can be designed to incorporate a partial Fc without a hinge and with a CH2 domain that is truncated but retains FcRn binding in order to confer longer terminal half-life on the construct. In yet another embodiment, the binding fusion polypeptide can be designed to incorporate a partial Fc without hinge but with a CH2 and CH3 domain, which can dimerize via the CH3 domain.

The dimerization polypeptide utilized in the fusion polypeptide can employ heterodimeric dimerization domains from a variety of sources. They include but are not limited to heterodimeric receptors that bind to growth factors (e.g. heregulin), neurotransmitters (e.g. γ-Aminobutyric acid), and other organic or inorganic small molecules (e.g. mineralocorticoid, glucocorticoid). Exemplary heterodimeric receptors are nuclear hormone receptors, erbB3 and erbB2 receptor complex, and G-protein-coupled receptors including but not limited to opioid, muscarinic, dopamine, serotonin, adenosine/dopamine, and GABAB families of receptors. For majority of the known heterodimeric receptors, their C-terminal sequences are found to mediate heterodimer formation. A compilation of known dimerization domains is provided on the following websites http://coiledcoils.chm.bris.ac.uk/ccplus/search/ and has been described in Testa et al., Nucleic Acid Research (2009) 37: D315-D322. Additionally, coiled coil structures were described in Vincent, T L, et al., Bioinformatics (2013) 29: 69-76; Armstrong et al., Bioinformatics (2011) 27: 1908-1914; Woolfson D N, Adv. Prot. Chem. (2005) 70: 79-112; Mason J M and Arndt K M, ChemBioChem (2004) 5: 170-176; each of which is incorporated by reference herein as if fully set forth.

In an embodiment, the dimerization polypeptide may include coiled coils from basic leucine zippers. The other basic leucine zippers may be TF6, CREB1, C/EBPct, Fos, or Jun, viral fusion polypeptides influenza hemagglutinin or HIV gp41, or other coiled coil domains APC or ProP. Dimerization domains may be thermo-sensitive dimeric coiled coils or more complex multimeric coil structures in recombinant proteins as a means to regulate enzyme activity both in vivo and in vitro.

Another exemplary class of heterodimerization sequences consists of amphiphilic peptides that adopt a coiled-coil helical structure. Well-characterized coiled-coil-containing proteins include members of the cytoskeletal family (e.g. α-keratin, vimentin), cytoskeletal motor family (e.g. myosin, kinesins, and dyneins), viral membrane proteins (e.g. membrane proteins of Ebola or HIV), DNA binding proteins, and cell surface receptors (e.g. GABAB receptors 1 and 2). Coiled-coil heterodimerization sequences of the present invention can be broadly classified into two groups, namely the left-handed and right-handed coiled coils. The left-handed coiled coils are characterized by a heptad repeat denoted “abcdefg” with the occurrence of apolar residues preferentially located at the first (a) and fourth (d) position. The residues at these two positions typically constitute a zig-zag pattern of “knobs and holes” that interlock with those of the other stand to form a tight-fitting hydrophobic core. In contrast, the second (b), third (c) and sixth (f) positions that cover the periphery of the coiled coil are preferably charged residues. Examples of charged amino acids include basic residues such as lysine, arginine, histidine, and acidic residues such as aspartate, glutamate, asparagine, and glutamine. Uncharged or apolar amino acids suitable for designing a heterodimeric coiled coil include but are not limited to glycine, alanine, valine, leucine, isoleucine, serine and threonine. While the uncharged residues typically form the hydrophobic core, inter-helical and intra-helical salt-bridge including charged residues even at core positions may be employed to stabilize the overall helical coiled-coiled structure (Burkhard et al. (2000) J. Biol. Chem. 275:11672-11677). Whereas varying lengths of coiled coil may be employed, the subject heterodimerization sequences preferably contain two to ten heptad repeats. More preferably, the heterodimerization sequences contain three to eight heptad repeats, even more preferably contain four to five heptad repeats.

In designing optimal coiled-coil heterodimerization sequences, a variety of existing computer software programs that predict the secondary structure of a peptide can be used. An illustrative computer analysis uses the COILS algorithm which compares an amino acid sequence with sequences in the database of known two-stranded coiled coils, and predicts the high probability coiled-coil stretches (Kammerer et al. (1999) Biochemistry 38:13263-13269).

In an embodiment, dimerization polypeptides may include dimerization domains other than coiled coils. Dimerization domains may be, but are not limited to, membrane dimerization domains, dimerization domains from transcription factors other than leucine zippers, G protein βγ complexes from heterotrimeric G protein complexes, TIM, ADH5, 14-3-3 proteins or their binding partners Bad or Bax, or other protein dimers. Membrane dimerization domains may be glycophorin A, receptor tyrosine kinases, or GPCRs. Dimerization domains from transcription factors other than leucine zippers may be nuclear receptors, an estrogen receptor, an androgen receptor, a glucocorticoid receptor, basic helix-loop-helix MyoD or c-Myc, helix-turn-helix LuxR, TetR, or cl.

Additionally, computer modeling and searching technologies further facilitates detection of heterodimerization sequences based on sequence homologies of common domains appeared in related and unrelated genes. Non-limiting examples of programs that allow homology searches are Blast (http://www.ncbi.nlm.nih.gov/BLAST/), Fasta (Genetics Computing Group package, Madison, Wis.), DNA Star, Clustlaw, TOFFEE, COBLATH, Genthreader, and MegAlign. Any sequence databases that contains DNA sequences corresponding to a target receptor or a segment thereof can be used for sequence analysis. Commonly employed databases include but are not limited to GenBank, EMBL, DDBJ, PDB, SWISS-PROT, EST, STS, GSS, and HTGS.

In some cases, dimerization is mediated by non-covalent interactions between fusion polypeptides. In some cases, dimerization is mediated by covalent interactions between fusion polypeptides. For example, covalent interactions can comprise one or more disulfide bonds formed between cysteine residues on two dimerization polypeptides.

In one aspect, the invention provides dimers of the disclosed fusion polypeptides. Dimers can be homodimers, in which each fusion polypeptide in the dimer is identical. Dimers can be heterodimers, in which each fusion polypeptide in the dimer is different.

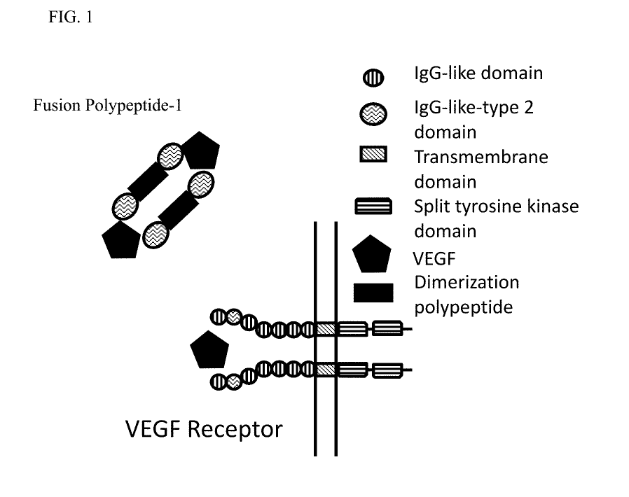

VEGF receptors comprise immunoglobulin domains. In some cases, wild-type VEGF receptors comprise an extracellular ligand binding domain comprising 7 immunoglobulin domains, a transmembrane spanning region, and an intracellular domain comprising a split tyrosine-kinase domain (see FIG. 1). The extracellular ligand binding domain is defined as the portion of a receptor that, in its native conformation in the cell membrane, is oriented extracellularly where it can contact with its cognate ligand. The extracellular ligand binding domain does not include the hydrophobic amino acids associated with the receptor's transmembrane domain or any amino acids associated with the receptor's intracellular domain. Generally, the intracellular or cytoplasmic domain of a receptor is usually composed of positively charged or polar amino acids (i.e. lysine, arginine, histidine, glutamic acid, aspartic acid). The preceding 15-30, predominantly hydrophobic or apolar amino acids (i.e. leucine, valine, isoleucine, and phenylalanine) comprise the transmembrane domain. The extracellular domain comprises the amino acids that precede the hydrophobic transmembrane stretch of amino acids. Usually the transmembrane domain is flanked by positively charged or polar amino acids such as lysine or arginine.

In some cases, the VEGF receptor immunoglobuline-like-type-2 domain is from a VEGF receptor from a mammal, such as a rat, human, mouse, dog, horse, cat, or sheep. In some cases, the VEGF receptor immunoglobuline-like-type-2 domain is from a VEGF receptor from an animal, such as a nematode, an ant, a bird, a whale, cnidarian, or a fish. In an exemplary embodiment, the VEGF receptor is from a human.

In some cases, the VEGF receptor immunoglobuline-like-type-2 domain is at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% identical to a VEGF receptor immunoglobuline-like-type-2 domain from a wild-type VEGF receptor. In some cases, the VEGF receptor immunoglobuline-like-type-2 domain is from a VEGF receptor is selected from the group consisting of Flt1, Kdr, and Flt-4. In some cases, the VEGF receptor is Flt1. In some cases the VEGF receptor is Kdr. In some cases the VEGF receptor is Flt-4. In some cases, the VEGF receptor is a human VEGF receptor. In some cases, the VEGF receptor is selected from the group consisting of SEQ IDs NO: 4-6.

In some cases, a VEGF receptor immunoglobulin-like-type 2 domain of the fusion polypeptide can be truncated. In some cases, the VEGF receptor immunoglobulin-like-type 2 domains of the fusion polypeptide comprise at least 70%, at least 75%, at least 80%, at least 85%, at least 90%, at least 95%, at least 96%, at least 97%, at least 98%, or at least 99% the length of a whole VEGF receptor immunoglobulin-like-type 2 domain. In some cases, the VEGF receptor immunoglobulin-like-type 2 domain is a human VEGF receptor. In some cases, the VEGF receptor immunoglobulin-like-type 2 domain of the fusion polypeptide is at least 70%, at least 75%, at least 80%, at least 85%, at least 90%, at least 95%, at least 96%, at least 97%, at least 98%, or at least 99% the length of a sequence selected from the group consisting of SEQ IDs NO: 4-6.

In some cases, the fusion polypeptide comprises two or more immunoglobulin-like-type 2 domains. For example, the fusion polypeptide can comprise two, three, four, five, six, seven, eight, nine, ten, or more immunoglobulin-like-type 2 domains. The two or more domains can be fused on either side of a dimerization polypeptide. For example, a first immunoglobulin-like-type 2 domain can be fused at the N-terminus of a dimerization polypeptide, and a second immunoglobulin-like-type 2 domain can be fused at the C-terminus of a dimerization polypeptide. The two or more immunoglobulin-like-type 2 domains can be derived from the same VEGF receptor. The two or more immunoglobulin-like-type 2 domains can be derived from distinct VEGF receptors. For example, a first immunoglobulin-like-type 2 domain can be derived from Flt1 and a second from Kdr.

The fusion polypeptide may comprise VEGF receptor immunoglobulin-like-type 2 domains connected directly to each other or to the dimerization polypeptide. The fusion polypeptide may comprise VEGF receptor immunoglobulin-like-type 2 domains connected to each other or to the dimerization polypeptide via spacers or linkers. In some cases, the spacers or linkers include portions or all of an antibody hinge region.

In some cases, the fusion polypeptide substantially lacks a VEGF receptor immunoglobulin-like-type 3 domain. For example, the fusion polypeptide may comprise no more than 60 amino acids, 55 amino acids, 50 amino acids, 45 amino acids, 40 amino acids, 35 amino acids, 30 amino acids, 25 amino acids, 20 amino acids, 15 amino acids, 10 amino acids, 5 amino acids of a VEGF receptor immunoglobulin-like-type 3 domain. In some cases, the fusion polypeptide lacks a sequence that is at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% identical to a VEGF receptor immunoglobulin-like type 3 domain. In some cases, the VEGF receptor is selected from the group consisting of Flt1, Kdr, and Flt-4. In some cases, the VEGF receptor is Flt1. In some cases the VEGF receptors is Kdr. In some cases the VEGF receptor is Flt-4. In some cases, the VEGF receptor immunoglobulin-like type 3 is a human VEGF receptor immunoglobulin-like type 3 domain. In some cases, the VEGF receptor is selected from the group consisting of SEQ IDs NO: 1-3. In some cases, the fusion polypeptide lacks SEQ ID NO: 8.

In some cases, the fusion polypeptide comprising two or more vascular endothelial growth factor (VEGF) receptor immunoglobulin-like-type-2 domains fused to a dimerization polypeptide, wherein a first homodimer of the fusion polypeptide binds VEGF with a higher affinity than a second homodimer of aflibercept (SEQ ID NO: 9) when measured by: (a) incubating the first homodimer and the second homodimer separately with immobilized VEGF to produce bound complexes; (b) washing the bound complexes to remove non-specific binding; and (c) performing enzyme-linked immunosorbent assay (ELISA) to assess an amount of the bound complexes after step (b)

In some cases, the binding affinity of the homodimer of the fusion polypeptides is higher than that of the fusion polypeptide oligomer. The homodimer of the fusion polypeptides can have a higher binding affinity for VEGF than wild-type VEGFR. The homodimer of the fusion polypeptides can have a higher binding affinity for VEGF than the homodimer of aflibercept (SEQ ID NO: 9).

Binding affinity can be observed or measured using art-recognized techniques including but not limited to ELISA, competitive ELISA, in vitro and in vivo neutralization assays (see Example 2).

In one example, binding affinity is measured by an ELISA assay. VEGF and a polypeptide can be incubated to achieve equilibrium binding. An antibody, directed to VEGF or the polypeptide, comprising an enzyme is incubated with the putative binding partner. After washing, the amount of protein complex is determined by incubating with the enzyme substrate and measuring the product. Exemplary methods for using ELISA to determine binding affinity can be found in Gan and Patel, Enzyme Immunoassay and Enzyme-Linked Immunosorbent Assay, Journal of Investigative Dermatology (2013), which is hereby incorporated by reference in its entirety.

In one example, binding affinity is measured by fluorescence resonance energy transfer (FRET). VEGF and the peptide of interest are labeled with a first and second fluorescent molecule, respectively. FRET relies on the principle that energy can be transferred between two light-sensitive molecules. A donor chromophore in an excited state can transfer energy to an acceptor chromophore through nonradiative dipole-dipole coupling. The efficiency of this transfer is proportional the sixth power of the distance of the two molecules, making FRET extremely sensitive to changes in distance between the two molecules. The equilibrium binding constant K_{d} can be determined by setting up a titration where the acceptor-tagged protein is added to the donor-tagged protein while the emission spectra are monitored. The ratio of donor-derived fluorescence to acceptor-derived fluorescence can be used to determine the binding affinity of the two proteins. For exemplary methods see Martin et al., Quantitative analysis of multi-protein interactions using FRET: Application to the SUMO pathway. Protein Science (2008), which is hereby incorporated by reference in its entirety.

In a second example, binding affinity can be measured by Surface Plasmon Resonance (SPR). SPR measures the change in the angle at which polarized light is reflected from a surface. The angle is related to the change in mass or layer thickness of the surface of a chip. Binding affinity can be measured using, for example, the ProteON XPR36 protein interaction array system (Bio-Rad).

In one aspect, the disclosure provides for a polynucleotide encoding any fusion polypeptide described herein. The polynucleotide can be, for example, the polynucleotide of SEQ ID NO: 7. A polynucleotide described herein can be obtained using chemical synthesis, molecular cloning or recombinant methods, DNA or gene assembly methods, artificial gene synthesis, PCR, or any combination thereof. Methods of chemical polynucleotide synthesis are well known in the art and need not be described in detail herein. One of skill in the art can use the sequences provided herein and a commercial DNA synthesizer to produce a desired DNA sequence. For preparing polynucleotides using recombinant methods, a polynucleotide comprising a desired sequence can be inserted into a suitable cloning or expression vector, and the cloning or expression vector in turn can be introduced into a suitable host cell for replication and amplification, as further discussed herein. Polynucleotides may be inserted into host cells by any means known in the art. Cells may be transformed by introducing an exogenous polynucleotide, for example, by direct uptake, endocytosis, transfection, F-mating, chemical transformation, or electroporation. Once introduced, the exogenous polynucleotide can be maintained within the cell as a non-integrated expression vector (such as a plasmid) or integrated into the host cell genome. The polynucleotide so amplified can be isolated from the host cell by methods well known within the art. Alternatively, nucleic acid amplification methods (e.g., PCR) allow reproduction of DNA sequences.

For recombinant expression of fusion polypeptide disclosed herein, the polynucleotide encoding it is isolated and inserted into a replicable vector for further cloning (amplification of the DNA) or for expression. In some embodiments, fusion polypeptide is cloned into the vector, and the wild-type sequence is mutated to produce a mutant fusion polypeptide expression vector, such as by directed mutation using methods known in the art (e.g. by PCR with a primer containing the desired mutation). DNA encoding wild-type and mutant fusion polypeptide is readily isolated and sequenced using conventional procedures (e.g., by using oligonucleotide probes and primers). Many vectors are available. The vector components generally include, but are not limited to, one or more of the following: a signal sequence, an origin of replication, one or more marker genes, an enhancer element, a promoter, and a transcription-termination sequence.

In general, and unless the expression vector is introduced into a host cell chromosome, both expression and cloning vectors contain a polynucleotide sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid, G418, kanamycin, and hygromycin. See U.S. Pat. No. 4,965,199.

A suitable selection gene for use in yeast is the trp1 gene present in the yeast plasmid YRp7 (Stinchcomb et al., Nature, 282:39 (1979)). The trp1 gene provides a selection marker for a mutant strain of yeast lacking the ability to grow in tryptophan, for example, ATCC No. 44076 or PEP4-1. Jones, Genetics, 85:12 (1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene. In addition, vectors derived from the 1.6-μm circular plasmid pKD1 can be used for transformation of Kluyveromyces yeasts. Alternatively, an expression system for large-scale production of recombinant calf chymosin was reported for K. lactis. Van den Berg, Bio/Technology, 8:135 (1990). Stable multi-copy expression vectors for secretion of mature recombinant human serum albumin by industrial strains of Kluyveromyces have also been disclosed. Fleer et al., Bio/Technology, 2: 968-975 (1991).

In some embodiments, the expression vector also comprises a nucleotide sequence encoding a detectable label. A detectable label may include, but is not limited to an enzyme, a transcription factor, a radioisotope binding protein, a fluorescent protein, or a fluorescent protein complex. In certain aspects, the fluorescent protein is a green fluorescent protein (GFP), cyan fluorescent protein (CFP), blue fluorescent protein (BFP), yellow fluorescent protein (YFP), red fluorescent protein (RFP), variants thereof, or various combinations thereof. In some embodiments, the detectable label is detectable by fluorescence, enzymatic activity, FRET, or NMR.

Expression and cloning vectors usually contain a promoter that is recognized by the host organism and is operably linked to fusion polypeptide-encoding polynucleotide. Promoters suitable for use with prokaryotic hosts include the phoA promoter, β-lactamase and lactose promoter systems, alkaline phosphatase, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the fusion polypeptide.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with eukaryotic hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other eukaryotic promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in eukaryotic expression are further described in EP 73,657. Eukaryotic enhancers also are advantageously used with eukaryotic promoters. Non-limiting examples of eukaryotic cells include yeast cells and mammalian cell lines.

Fusion polypeptide protein expression from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), heterologous mammalian promoters, e.g., the actin promoter or an immunoglobulin promoter, and heat-shock promoters, provided such promoters are compatible with the host cell systems. The early and late promoters of the SV40 virus are conveniently obtained as an SV40 restriction fragment that also contains the SV40 viral origin of replication. The immediate early promoter of the human cytomegalovirus is conveniently obtained as a Hin III E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature, 297:598-601 (1982) on expression of human β-interferon cDNA in mouse cells under the control of a thymidine kinase promoter from herpes simplex virus. Alternatively, the rous sarcoma virus long-terminal repeat can be used as the promoter.

Transcription of a DNA encoding an fusion polypeptide by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early-promoter enhancer, the polyoma enhancer on the late side of the replication origin, and adenovirus enhancers. See also Yaniv, Nature, 297:17-18 (1982) on enhancing elements for activation of eukaryotic promoters. The enhancer may be spliced into the vector at a position 5′ or 3′ to the fusion polypeptide-encoding sequence, but is preferably located at a site 5′ from the promoter.

Expression vectors used in eukaryotic host cells (for example, yeast, fungi, insect, plant, animal, human, or nucleated cells from other multicellular organisms) will also contain sequences necessary for the termination of transcription and for stabilizing the mRNA. Such sequences are commonly available from the 5′ end, occasionally 3′ end, of untranslated regions of eukaryotic or viral DNAs or cDNAs. These regions contain nucleotide segments transcribed as polyadenylated fragments in the untranslated portion of the mRNA encoding the fusion polypeptide. One useful transcription termination component is the bovine growth hormone polyadenylation region. See WO 1994/11026 and the expression vector disclosed therein.

### Host Cells

In one aspect, the disclosure provides a host cell comprising an expression vector comprising a nucleotide sequence encoding a fusion polypeptide disclosed herein, such as expression vectors described herein. In some embodiments, the expression vector is extrachromosomal, such as a plasmid. In some embodiments, the host cell comprises a stably integrated transgenic nucleotide sequence encoding the fusion polypeptide. Under suitable conditions, the host cell actively expresses the fusion polypeptide. Conditions suitable for expression depend on a number of factors known in the art, such as growth conditions for the cells and the activity of the promoter driving expression, which may be constitutively active, active in specific cell types, inducible in response to the presence of an inducing agent, or any other promoter described herein or known in the art.

In some embodiments the host cell expresses a selectable marker. Examples of selectable markers are provided herein, and may be expressed from the expression vector encoding the fusion polypeptide, or separately, such as from another expression vector which may or may not be integrated into the host cell genome. In some embodiments, the host cell expresses a detectable label. Examples of detectable labels are provided herein, and may be expressed from the expression vector encoding the fusion polypeptide, or separately, such as from another expression vector which may or may not be integrated into the host cell genome.

Suitable host cells for cloning or expressing the DNA in the vectors herein are the prokaryote, yeast, or higher eukaryote cells described herein and otherwise known in the art. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. One exemplary E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for fusion polypeptide-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of fusion polypeptide include cells derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK, ATCC CCL 10); Chinese hamster ovary cells/−DHFR (CHO, Urlaub et al., Proc. Natl. Acad. Sci. USA, 77:4216 (1980), including DG44 (Urlaub et al., Som. Cell and Mol. Gen., 12: 555-566 (1986)) and DP12 cell lines); mouse sertoli cells (TM4, Mather, Biol. Reprod., 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci., 383:44-68 (1982)); MRC 5 cells; FS4 cells; and a human hepatoma line (Hep G2).

A wide variety of additional cell lines for various tissue culture applications, gene expression, and assays are known in the art. Examples of cell lines include, but are not limited to, C8161, CCRF-CEM, MOLT, mIMCD-3, NHDF, HeLa-S3, Huhl, Huh4, Huh7, HUVEC, HASMC, HEKn, HEKa, MiaPaCell, Pancl, PC-3, TF1, CTLL-2, C1R, Rath, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRCS, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalc1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)).

Host cells can be transfected with one or more of the above-described expression or cloning vectors for fusion polypeptide expression and cultured in conventional nutrient media modified as appropriate for inducing promoters, selecting transformants, or amplifying the genes encoding the desired sequences.

Host cells transfected with expression vectors for the expression of a fusion polypeptide herein may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described, for example, in Ham et al., Meth. Enz. 58:44 (1979); Barnes et al., Anal. Biochem. 102:255 (1980); U.S. Pat. Nos. 4,767,704; 4,657,866; 4,927,762; 4,560,655; or 5,122,469; WO 1990/03430; WO 1987/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Several transfection protocols are known in the art, and are reviewed in Kaufman R. J., et al., Nucleic Acids Res. 19:4485, 1991. The transfection protocol chosen will depend on the host cell type and the nature of the expression vector, and can be chosen based upon routine experimentation. Typically, a transfection protocol includes introducing an expression vector into a suitable host cell, and then identifying and isolating host cells which have incorporated the heterologous DNA in a stable, expressible manner.

One commonly used method of introducing heterologous DNA is calcium phosphate precipitation, for example, as described by Wigler et al. (Proc. Natl. Acad. Sci. USA 77:3567, 1980). DNA introduced into a host cell by this method frequently undergoes rearrangement, making this procedure useful for cotransfection of independent genes.

Polyethylene-induced fusion of bacterial protoplasts with mammalian cells (Schaffner et al., Proc. Natl. Acad. Sci. USA 77:2163, 1980) is another useful method of introducing heterologous DNA. Protoplast fusion protocols frequently yield multiple copies of the plasmid DNA integrated into the mammalian host cell genome. This technique typically requires the selection and amplification marker to be on the same plasmid as the gene of interest.

Electroporation can also be used to introduce DNA directly into the cytoplasm of a host cell, as described by Potter et al. (Proc. Natl. Acad. Sci. USA 81:7161, 1988) or Shigekawa and Dower (BioTechniques 6:742, 1988). In general, electroporation does not require the selection marker and the gene of interest to be on the same plasmid.

Several reagents useful for introducing heterologous DNA into a mammalian cell have been described. These include Lipofectin® Reagent and Lipofectamine™ Reagent (Gibco BRL, Gaithersburg, Md.). Both of these reagents are commercially available reagents used to form lipid-nucleic acid complexes (or liposomes) which, when applied to cultured cells, facilitate uptake of the polynucleotide into the cells.

Transfection of cells with heterologous DNA and selection for cells that have taken up the heterologous DNA and express the selectable marker results in a pool of transfected cells. Individual cells in these pools may vary in the amount of DNA incorporated and in the chromosomal location of the transfected DNA. After repeated passage, pools may lose the ability to express the heterologous protein. To generate stable cell lines, individual cells can be isolated from the pools and cultured (a process referred to as cloning). In some instances, the pools themselves may be stable (e.g., production of the heterologous recombinant protein remains stable). The ability to select and culture such stable pools of cells would be desirable as it would allow rapid production of relatively large amounts of recombinant protein from mammalian cells.

### Methods of Administration of Fusion Polypeptides Disclosed Herein

The invention comprises methods of treatment comprising administering to a subject an effective amount of an agent of the invention. In an exemplary aspect, the agent is substantially purified (e.g., substantially free from substances that limit its effect or produce undesired side-effects). The agent can be a fusion polypeptide disclosed herein. The agent can be a dimer of a fusion polypeptide disclosed herein. The subject is preferably an animal, e.g., such as cows, pigs, horses, chickens, cats, dogs, etc., and is preferably a mammal, and most preferably human.

In some cases, the pharmaceutical compositions of the invention are administered to the area in need of treatment by injection or topical administration. Topical drug delivery is the most common treatment for diseases or disorders of the anterior segment of the eye, including, for example, corneal diseases, uveitis, and glaucoma. Topical delivery can be a safer and more convenient delivery method for patients, and can reduce the risk of many side effects observed in systemic treatment regimens. Topical administration of an angiogenesis inhibitor to the eye or cornea can be an effective treatment for treating neovascularization and/or inflammation. A method of administering the pharmaceutical compositions of the invention to the eye is by eye drops comprising a fusion polypeptide disclosed herein.

In various embodiments, the pharmaceutical compositions of the invention are administered to the area in need of treatment by subconjunctival administration. One exemplary method of subconjunctival administration to the eye is by injectable formulations comprising a fusion polypeptide disclosed herein. Another exemplary method of subconjunctival administration is by implantations comprising slow releasing fusion polypeptide or dimer disclosed herein.

In various embodiments, the pharmaceutical compositions of the invention are administered by intravenous or intraperitoneal injection. One exemplary method of intravenous or intraperitoneal administration by injectable formulations comprising a fusion polypeptide or dimer disclosed herein.

The invention provides methods for treating or preventing an inflammatory disease, autoimmune disease, complement-related disease, ocular disease, and cancer. In some embodiments, the invention provides a method of treating a subject with an inflammatory disease, autoimmune disease, complement-related disease, ocular disease, and/or cancer, comprising administering to the subject an effective amount of any fusion polypeptide described herein. In some embodiments, the method further comprises administering to the individual an effective amount of at least one additional therapeutic agent, e.g., as described below. In some embodiments, the invention provides a fusion polypeptide for use in inhibiting binding of a VEGF to a VEGFR. In some embodiments, the invention provides a fusion polypeptide for use in inhibiting binding of a VEGF to a VEGFR in a subject comprising administering to the subject an effective amount of the fusion polypeptide to inhibit binding of a VEGF to a VEGFR. In some embodiments, the invention provides a fusion polypeptide for use in inhibiting VEGF signaling pathway (e.g., inhibition of VEGF activity) in a subject comprising administering to the subject an effective amount of the fusion polypeptide to VEGF signaling pathway (e.g., inhibition of VEGF activity). A “subject” according to any of the above embodiments is preferably human.

An inflammatory disease that can be treated or prevented by the fusion polypeptides described herein include, but is not limited to, macular degeneration (e.g., age-related macular degeneration), acute myocardial infarction (AMI), atherosclerosis, glomernephritis, asthma, and multiple sclerosis. An autoimmune disease that can be treated or prevented by the fusion polypeptides described herein include, but is not limited to, Alzheimer's disease, autoimmune uveitis, systemic lupus erythematosus (SLE), lupus nephritis, ulcerative colitis, inflammatory bowel disease, Crohn's disease, adult respiratory distress syndrome (ARDS), multiple sclerosis, diabetes mellitus, Huntington's disease, Parkinson's disease, rheumatoid arthritis, juvenile rheumatoid arthritis, osteoarthritis, psoriatic arthritis, CNS inflammatory disorders, myasthenia gravis, glomerulonephritis, and autoimmune thrombocytopenia. A complement-related disease that can be treated or prevented by the fusion polypeptides described herein include, but is not limited to, aneurysm, atypical hemolytic uremic syndrome, thrombotic thrombocytopenic purpura, idiopathic thrombocytopenic purpura, AMD, spontaneous fetal loss, recurrent fetal loss, traumatic brain injury, psoriasis, autoimmune hemolytic anemia, hereditary angioedema, stroke, hemorrhagic shock, septic shock, complication from surgery such as coronary artery bypass graft (CABG) surgery, pulmonary complications such as chronic obstructive pulmonary disease (COPD), ischemia-reperfusion injury, organ transplant rejection, and multiple organ failure. In some embodiments, the cancer that can be treated or prevented by the fusion polypeptides described herein includes colorectal cancer, non-small cell lung cancer, lymphoma, leukemia, adenocarcinoma, glioblastoma, kidney cancer, gastric cancer, prostate cancer, retinoblastoma, ovarian cancer, endometrial cancer, and breast cancer. In a further embodiment, any of the cancers disclosed herein that can be treated or prevented by the fusion polypeptides described herein is metastatic. An ocular disease that can be treated or prevented by the fusion polypeptides described herein include, but is not limited to, wet age-related macular degeneration, dry age-related macular degeneration, diabetic retinopathy, diabetic retinal edema, diabetic macular edema, retrolental fibroplasias, retinal central occlusion, retinal vein occlusion, ischemic retinopathy, hypertensive retinopathy, uveitis (e.g., anterior, intermediate, posterior, or panuveitis), Behcet's disease, Biett's crystalline dystrophy, blepharitis, glaucoma (e.g., open-angle glaucoma), neovascular glaucoma, neovascularization of the cornea, choroidal neovascularization (CNV), subretinal neovascularization, corneal inflammation, and complications from corneal transplantation.

The fusion polypeptides and compositions described herein are particularly useful for treating macular degeneration such as AMD. AMD is the leading cause of blindness and visual impairment among the elderly (>50 years) in the United States and other developed countries (Bird, A. C., (2010). J. Clin. Invest., 120(9): 3033-3041). AMD is broadly classified into two types, a wet form and a dry form, with the dry form constituting up to 80-90% of all AMD cases. Dry AMD (non-exudative) is a form of AMD in which cellular debris called drusen accumulates between the retina and the choroid. Dry AMD has three stages, early, intermediate, and advanced, and is characterized by the presence of macular drusen. In advanced dry AMD, central geopraphic atrophy occurs resulting loss of vision in the center of the eye. The wet (exudative or neovascular) form AMD is the more severe form in which abnormal blood vessels (choroidal neovascularization, CNV) grow up from the choroid through Bruch's membrane behind the macula, resulting in rapid vision loss. In recent years, increasing evidence has indicated that complement activation plays a major role in pathogenesis of AMD (Issa, P. C., et al, (2011), Graefes. Arch. Clin. Exp. Ophthalmol., 249: 163-174). It is generally accepted that dry AMD can progress to wet AMD. The present invention provides methods of treating AMD (such as wet or dry forms of AMD) by administering an effective amount of a composition comprising a fusion polypeptide as described herein. In some embodiments, the invention provides methods of treating or preventing one or more aspects or symptoms of AMD, including, but not limited to, formation of ocular drusen, inflammation in the eye or eye tissue, loss of photoreceptor cells, loss of vision (including for example visual acuity and visual field), neovascularization, subretinal hemorrhage, retinal detachment, blood vessel leakage and any other AMD related aspects.

In a further aspect, the invention provides for the use of a fusion polypeptide in the manufacture or preparation of a medicament. In some embodiments, the medicament is for treatment of an inflammatory disease, autoimmune disease, complement-related disease, ocular disease, and cancer. In some embodiments, the invention provides a fusion polypeptide for the manufacture of a medicament for use in inhibiting binding of a complement protein to a complement regulating protein. In some embodiments, the invention provides a fusion polypeptide for the manufacture of a medicament for use in inhibiting binding of a VEGF to a VEGFR. In some embodiments, the invention provides a fusion polypeptide for the manufacture of a medicament for use in inhibiting complement activation and VEGF signaling pathway (e.g., inhibition of VEGF activity) in a subject comprising administering to the subject an effective amount of the fusion polypeptide to inhibit complement activation and VEGF signaling pathway (e.g., inhibition of VEGF activity). A “subject” according to any of the above embodiments is preferably human. In some embodiments, the medicament is used for treatment of an inflammatory disease including, but not limited to, macular degeneration (e.g., age-related macular degeneration), acute myocardial infarction (AMI), atherosclerosis, glomernephritis, asthma, and multiple sclerosis. In some embodiments, the medicament is used for treatment of an autoimmune disease including, but not limited to, Alzheimer's disease, autoimmune uveitis, systemic lupus erythematosus (SLE), lupus nephritis, ulcerative colitis, inflammatory bowel disease, Crohn's disease, adult respiratory distress syndrome (ARDS), multiple sclerosis, diabetes mellitus, Huntington's disease, Parkinson's disease, rheumatoid arthritis, juvenile rheumatoid arthritis, osteoarthritis, psoriatic arthritis, CNS inflammatory disorders, myasthenia gravis, glomerulonephritis, and autoimmune thrombocytopenia. In some embodiments, the medicament is used for treatment of a complement-related disease including, but not limited to, aneurysm, atypical hemolytic uremic syndrome, thrombotic thrombocytopenic purpura, idiopathic thrombocytopenic purpura, AMD, spontaneous fetal loss, recurrent fetal loss, traumatic brain injury, psoriasis, autoimmune hemolytic anemia, hereditary angioedema, stroke, hemorrhagic shock, septic shock, complication from surgery such as coronary artery bypass graft (CABG) surgery, pulmonary complications such as chronic obstructive pulmonary disease (COPD), ischemia-reperfusion injury, organ transplant rejection, and multiple organ failure. In some embodiments, the cancer that can be treated or prevented by the fusion polypeptides described herein includes colorectal cancer, metastatic colorectal cancer, non-small cell lung cancer, lymphoma, leukemia, adenocarcinoma, glioblastoma, kidney cancer, metastatic kidney cancer, gastric cancer, prostate cancer, retinoblastoma, ovarian cancer, endometrial cancer, and breast cancer. In other embodiments, the medicament is used for treatment of an ocular disease including, but not limited to, wet age-related macular degeneration, dry age-related macular degeneration, diabetic retinopathy, diabetic retinal edema, diabetic macular edema, retrolental fibroplasias, retinal central occlusion, retinal vein occlusion, ischemic retinopathy, hypertensive retinopathy, uveitis (e.g., anterior, intermediate, posterior, or panuveitis), Behcet's disease, Biett's crystalline dystrophy, blepharitis, glaucoma (e.g., open-angle glaucoma), neovascular glaucoma, neovascularization of the cornea, choroidal neovascularization (CNV), subretinal neovascularization, corneal inflammation, and complications from corneal transplantation.

By way of example, but not limitation, the method of the invention may be useful in treating clinical conditions that are characterized by vascular permeability, edema or inflammation such as brain edema associated with injury, stroke or tumor; edema associated with inflammatory disorders such as psoriasis or arthritis, including rheumatoid arthritis; asthma; generalized edema associated with burns; ascites and pleural effusion associated with tumors, inflammation or trauma; chronic airway inflammation; capillary leak syndrome; sepsis; kidney disease associated with increased leakage of protein; and eye disorders such as age related macular degeneration and diabetic retinopathy.

### Pharmaceutical Compositions for Ophthalmic Administration

Pharmaceutical compositions useful in the practice of the method of the invention include a therapeutically effective amount of an active agent with a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the therapeutic is administered. Examples of suitable pharmaceutical carriers are described in “Remington's Pharmaceutical Sciences” by E. W. Martin. In an exemplary embodiment, the composition is formulated in accordance with routine procedures as a pharmaceutical composition adapted for topical administration to human beings. Such pharmaceutical compositions may be liquid, gel, ointment, salve, slow release formulations or other formulations suitable for ophthalmic administration. The composition comprises an effective amount of a fusion polypeptide or dimer disclosed herein and, optionally, at least one ophthalmically acceptable excipient, wherein the excipient is able to reduce a rate of removal of the composition from the eye by lacrimation, such that the composition has an effective residence time in the eye of about 2 hours to about 24 hours.

In various embodiments, compositions of the invention can comprise a liquid comprising an active agent in solution, in suspension, or both. The term “suspension” herein includes a liquid composition wherein a first portion of the active agent is present in solution and a second portion of the active agent is present in particulate form, in suspension in a liquid matrix. As used herein, liquid compositions include gels.

In some cases, the liquid composition is aqueous. Alternatively, the composition can take form of an ointment. In an exemplary embodiment, the composition is an in situ gellable aqueous composition, for example an in situ gellable aqueous solution. Such a composition can comprise a gelling agent in a concentration effective to promote gelling upon contact with the eye or lacrimal fluid in the exterior of the eye. Suitable gelling agents non-restrictively include thermosetting polymers such as tetra-substituted ethylene diamine block copolymers of ethylene oxide and propylene oxide (e.g., poloxamine 1307); polycarbophil; and polysaccharides such as gellan, carrageenan (e.g., kappa-carrageenan and iota-carrageenan), chitosan and alginate gums. The phrase “in situ gellable” includes not only liquids of low viscosity that can form gels upon contact with the eye or with lacrimal fluid in the exterior of the eye, but also more viscous liquids such as semi-fluid and thixotropic gels that exhibit substantially increased viscosity or gel stiffness upon administration to the eye or area surrounding the eye.

Aqueous compositions of the invention have ophthalmically compatible pH and osmolality. In some cases, these compositions incorporate means to inhibit microbial growth, for example through preparation and packaging under sterile conditions and/or through inclusion of an antimicrobially effective amount of an ophthalmically acceptable preservative. Suitable preservatives non-restrictively include mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof; phenoxyethanol; chlorophenoxyethanol; phenoxypropanol; chlorobutanol; chlorocresol; phenylethyl alcohol; disodium EDTA; and sorbic acid and salts thereof.

The composition can comprise an ophthalmic depot formulation comprising an active agent for subconjunctival administration. The ophthalmic depot formulation comprises microparticles of essentially pure active agent, e.g., a fusion polypeptide or dimer disclosed herein. The microparticles comprising a fusion polypeptide or dimer disclosed herein can be embedded in a biocompatible pharmaceutically acceptable polymer or a lipid encapsulating agent. The depot formulations may be adapted to release all of substantially all the active material over an extended period of time. The polymer or lipid matrix, if present, may be adapted to degrade sufficiently to be transported from the site of administration after release of all or substantially all the active agent. The depot formulation can be liquid formulation, comprising a pharmaceutical acceptable polymer and a dissolved or dispersed active agent. Upon injection, the polymer forms a dot at the injections site, e.g. by gelifying or precipitating.

The composition can comprise a solid article that can be inserted in a suitable location in the eye, such as between the eye and eyelid or in the conjunctival sac, where the article releases the active agent. Release from such an article is in some cases to the cornea, either via lacrimal fluid that bathes the surface of the cornea, or directly to the cornea itself, with which the solid article is generally in intimate contact. Solid articles suitable for implantation in the eye in such fashion generally comprise polymers and can be bioerodible or non-bioerodible. Bioerodible polymers that can be used in preparation of ocular implants carrying a fusion polypeptide or dimer disclosed herein in accordance with the present invention include without restriction aliphatic polyesters such as polymers and copolymers of poly(glycolide), poly(lactide), poly(ε-caprolactone), poly(hydroxybutyrate) and poly(hydroxyvalerate), polyamino acids, polyorthoesters, polyanhydrides, aliphatic polycarbonates and polyether lactones. Illustrative of suitable non-bioerodible polymers are silicone elastomers.

The active agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

### Pharmaceutical Compositions for Injection.

In some embodiments, the invention provides a pharmaceutical composition for injection containing at least one fusion polypeptide or dimer of the present invention and a pharmaceutical excipient suitable for injection. Injection can provide for local or systemic administration of an agent.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Administration of Pharmaceutical Compositions

In some embodiments, a given dosing schedule comprising one or more administrations of a fusion polypeptide or dimer as described herein may be repeated on a daily, weekly, biweekly, monthly, bimonthly, annually, semi-annually, or any other period as may be determined by a medical professional. A repeated dosing schedule may be repeated for a fixed period of time determined at the start of the schedule; may be terminated, extended, or otherwise adjusted based on a measure of therapeutic effect, such as a level of reduction in the presence of detectable disease tissue (e.g. a reduction of at least 50%, 60%, 70%, 80%, 90%, 95%, 99%, or 100%); or may be terminated, extended, or otherwise adjusted for any other reason as determined by a medical professional.

A fusion polypeptide or dimer disclosed herein can be administered as part of a combination treatment, wherein fusion polypeptide or dimer is administered with one or more additional therapeutic agents. Such one or more additional agents can be administered simultaneously or separately with respect to the fusion polypeptide. Administration in combination utilizing one or more additional agents includes, for example, simultaneous administration of two agents in the same dosage form, simultaneous administration in separate dosage forms, and separate administration. For example, multiple therapeutic agents can be formulated together in the same dosage form and administered simultaneously. Alternatively multiple therapeutic agents can be simultaneously administered, wherein both the agents are present in separate formulations. In another alternative, a fusion polypeptide or dimer of the present invention can be administered just followed by one or more additional agents, or vice versa. In the separate administration protocol, a fusion polypeptide or dimer of the present invention and one or more additional agents may be administered a few minutes apart, or a few hours apart, or a few days apart. The term “combination treatments” also embraces the administration of the fusion polypeptides as described herein in further combination with other biologically active compounds or ingredients and non-drug therapies (e.g., surgery or radiation treatment).

Administration of the fusion polypeptide or dimer of the present invention can be effected by any method that enables delivery of the fusion polypeptide or dimer to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent. Fusion polypeptides can also be administered intraadiposally or intrathecally. An effective amount of a fusion polypeptide or dimer of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer. Sequential or substantially simultaneous administration of a fusion polypeptide, and/or any additional therapeutic agent can be effected by any appropriate route as noted above and including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection.

Methods of determining the most effective means and dosage of administration are well known to those of skill in the art and will vary with the composition used for therapy, the purpose of the therapy, the target cell or tissue being treated, and the subject being treated. Single or multiple administrations (e.g. about or more than about 1, 2, 3, 4, 5, 6, 7, 8, 9, 10, 11, 12, 13, 14, 15, 16, 17, 18, 19, 20, 25, 30, or more doses) can be carried out with the dose level and pattern being selected by the treating physician.

A fusion polypeptide or dimer may be administered in any suitable amount, and in the order disclosed herein. In some embodiments, a fusion polypeptide or dimer is administered to a subject within a range of about 0.1 mg/kg-50 mg/kg per day, such as about, less than about, or more than about, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 25 mg/kg, 30 mg/kg, 35 mg/kg, 40 mg/kg, 45 mg/kg, or 50 mg/kg per day. In some embodiments, a fusion polypeptide or dimer is administered to a subject within a range of about 0.1 mg/kg-400 mg/kg per week, such as about, less than about, or more than about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 100 mg/kg, 150 mg/kg, 200 mg/kg, 250 mg/kg, 300 mg/kg, 350 mg/kg, or 400 mg/kg per week. In some embodiments, a fusion polypeptide or dimer is administered to a subject within a range of about 0.1 mg/kg-1500 mg/kg per month, such as about, less than about, or more than about 50 mg/kg, 100 mg/kg, 150 mg/kg, 200 mg/kg, 250 mg/kg, 300 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, or 1000 mg/kg per month. In some embodiments, a fusion polypeptide or dimer is administered to a subject within a range of about 0.1 mg/m2-200 mg/m2 per week, such as about, less than about, or more than about 5 mg/m2, 10 mg/m2, 15 mg/m2, 20 mg/m2, 25 mg/m2, 30 mg/m2, 35 mg/m2, 40 mg/m2, 45 mg/m2, 50 mg/m2, 55 mg/m2, 60 mg/m2, 65 mg/m2, 70 mg/m2, 75 mg/m2, 100 mg/m2, 125 mg/m2, 150 mg/m2, 175 mg/m2, or 200 mg/m2 per week. The target dose may be administered in a single dose. Alternatively, the target dose may be administered in about or more than about 1, 2, 3, 4, 5, 6, 7, 8, 9, 10, 11, 12, 13, 14, 15, 16, 17, 18, 19, 20, 25, 30, or more doses. For example, a dose of about 20 mg/kg per week may be delivered weekly at a dose of about 20 mg/kg, or may be delivered at a dose of about 6.67 mg/kg administered on each of three days over the course of the week, which days may or may not be consecutive. The administration schedule may be repeated according to any prescribed regimen, including any administration schedule described herein. In some embodiments, a fusion polypeptide or dimer is administered to a subject in the range of about 0.1 mg/m²-500 mg/m², such as about, less than about, or more than about 5 mg/m², 10 mg/m², 15 mg/m², 20 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², 100 mg/m², 130 mg/m², 135 mg/m², 155 mg/m², 175 mg/m², 200 mg/m², 225 mg/m², 250 mg/m², 300 mg/m², 350 mg/m², 400 mg/m², 420 mg/m², 450 mg/m², or 500 mg/m².

An exemplary dosing regimen comprises administering an initial dose of a fusion polypeptide of about 2 mg/kg, followed by a weekly maintenance dose of about 1 mg/kg every other week. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg, about 100 μg/kg, about 300 μg/kg, about 1 mg/kg, and about 2/mg/kg) may be used. In certain embodiments, dosing frequency is three times per day, twice per day, once per day, once every other day, once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the fusion polypeptide administered, can vary over time independently of the dose used.

Dosages for a particular fusion polypeptide may be determined empirically in individuals who have been given one or more administrations of fusion polypeptide. Individuals are given incremental doses of a fusion polypeptide. To assess efficacy of a fusion polypeptide, a clinical symptom of an inflammatory disease (such as AMD) can be monitored.

Administration of a fusion polypeptide according to the methods of the invention can be continuous or intermittent, depending, for example, on the recipient's physiological condition, whether the purpose of the administration is therapeutic or prophylactic, and other factors known to skilled practitioners. The administration of a fusion polypeptide may be essentially continuous over a preselected period of time or may be in a series of spaced doses, e.g., either during or after development of an inflammatory disease (such as AMD).

Guidance regarding particular dosages and methods of delivery is provided in the literature; see, for example, U.S. Pat. Nos. 4,657,760; 5,206,344; or 5,225,212. It is within the scope of the invention that different formulations will be effective for different treatments and different diseases or disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

In some embodiments, a fusion polypeptide or dimer and/or any additional therapeutic compound of the invention is administered in multiple doses. Dosing may be about once, twice, three times, four times, five times, six times, or more than six times per day. Dosing may be about once a month, once every two weeks, once a week, or once every other day.

Administration of the fusion polypeptides of the invention may continue as long as necessary. In some embodiments, a fusion polypeptide or dimer of the invention is administered for more than 1, 2, 3, 4, 5, 6, 7, 14, or 28 days. In some embodiments, a fusion polypeptide or dimer of the invention is administered for less than 28, 14, 7, 6, 5, 4, 3, 2, or 1 day. In some embodiments, a fusion polypeptide or dimer of the invention is administered chronically on an ongoing basis, e.g., for the treatment of chronic effects.

When a combination treatment of the invention is administered as a composition that comprises one or more agents, and one agent has a shorter half-life than another agent, the unit dose forms may be adjusted accordingly.

The combination treatments according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the agent selected, the route of administration, the form in which the compound is administered, the subject to be treated, the body weight of the subject to be treated, and the preference and experience of the attending physician.

A pharmaceutical composition of the present invention typically contains an active ingredient (e.g., a fusion polypeptide or dimer disclosed herein, and one or more pharmaceutically acceptable excipients, carriers, including but not limited inert solid diluents and fillers, diluents, sterile aqueous solution and various organic solvents, permeation enhancers, solubilizers and adjuvants.

Described below are non-limiting exemplary pharmaceutical compositions and methods for preparing the same.

In another aspect of the present invention, methods are provided for treating ophthalmic disease by applying one or more of the subject combination treatments to the eye of a subject. Methods are further provided for administering the combination treatments of the present invention via eye drop, intraocular injection, intravitreal injection, topically, or through the use of a drug eluting device, microcapsule, implant, or microfluidic device. In some cases, combination treatments are administered with a carrier or excipient that increases the intraocular penetrance of the compound such as an oil and water emulsion with colloid particles having an oily core surrounded by an interfacial film.

In some cases, the colloid particles include at least one cationic agent and at least one non-ionic surfactant such as a poloxamer, tyloxapol, a polysorbate, a polyoxyethylene castor oil derivative, a sorbitan ester, or a polyoxyl stearate. In some cases, the cationic agent is an alkylamine, a tertiary alkyl amine, a quarternary ammonium compound, a cationic lipid, an amino alcohol, a biguanidine salt, a cationic compound or a mixture thereof. In some cases the cationic agent is a biguanidine salt such as chlorhexidine, polyaminopropyl biguanidine, phenformin, alkylbiguanidine, or a mixture thereof. In some cases, the quaternary ammonium compound is a benzalkonium halide, lauralkonium halide, cetrimide, hexadecyltrimethylammonium halide, tetradecyltrimethylammonium halide, dodecyltrimethylammonium halide, cetrimonium halide, benzethonium halide, behenalkonium halide, cetalkonium halide, cetethyldimonium halide, cetylpyridinium halide, benzododecinium halide, chlorallyl methenamine halide, r-myristylalkonium halide, stearalkonium halide or a mixture of two or more thereof. In some cases, cationic agent is a benzalkonium chloride, lauralkonium chloride, benzododecinium bromide, benzethenium chloride, hexadecyltrimethylammonium bromide, tetradecyltrimethylammonium bromide, dodecyltrimethylammonium bromide or a mixture of two or more thereof. In some cases, the oil phase is mineral oil and light mineral oil, medium chain triglycerides (MCT), coconut oil; hydrogenated oils comprising hydrogenated cottonseed oil, hydrogenated palm oil, hydrogenate castor oil or hydrogenated soybean oil; polyoxyethylene hydrogenated castor oil derivatives comprising poluoxyl-40 hydrogenated castor oil, polyoxyl-60 hydrogenated castor oil or polyoxyl-100 hydrogenated castor oil.

The compounds or pharmaceutical compositions of the present invention can be used in combination with an amount of one or more substances selected from anti-angiogenesis agents, signal transduction inhibitors, and antiproliferative agents.

For in vivo administration of the fusion polypeptides described herein, normal dosage amounts may vary from about 10 ng/kg up to about 100 mg/kg of an individual's body weight or more per day, in some cases about 1 mg/kg/day to 10 mg/kg/day, depending upon the route of administration. For repeated administrations over several days or longer, depending on the severity of the disease or disorder to be treated, the treatment is sustained until a desired suppression of symptoms is achieved.

As an example, the fusion polypeptide disclosed herein is administered to a patient with Neovascular (Wet) Age-Related Macular Degeneration (AMD). The fusion polypeptide is administered by opthalmic intravitreal injection. The dose is between 0.1 and 0.4 mg, preferably about 2 mg. Administration can be every 2 weeks, every 4 weeks, or every 8 weeks. After one month, two months, three months, or four months, the dosing frequency can changed. In some cases, the dosing frequency is changed to every 2 weeks, every 4 weeks, or every 8 weeks.

### Kits

The invention also provides an article of manufacture comprising packaging material and a pharmaceutical agent contained within the packaging material, wherein the pharmaceutical agent comprises at least one fusion polypeptide or dimer of the invention and wherein the packaging material comprises a label or package insert that indicates that the fusion polypeptide or dimer can be used for treating eye injury. The kit can comprise a composition comprising a fusion polypeptide or dimer and one or more other components such as, for example, components to be combined prior to use either by a health care professional or by the subject. In one embodiment, the fusion polypeptide or dimer is combined with one or more components that can comprise, for example, a solution included in the kit to reconstitute a fusion polypeptide or dimer in the form of an ophthalmical composition suitable for topical or subconjunctival administration to a human or animal. Kit components can comprise, for example, normal saline solutions and/or solutions comprising one or more suitable pharmaceutical carriers, stabilizers, additives, or buffers. In some cases the kit comprises instructions for treatment or administration regimens and/or instructions for preparing or reconstituting a fusion polypeptide or dimer for use. The instructions can be in writing on paper, on computer media of any suitable type, as audiovisual materials including, for example, CD or DVD, or any other suitable format.

Other features of the invention will become apparent in the course of the following descriptions of exemplary embodiments which are given for illustration of the invention and are not intended to be limiting thereof.

### EXAMPLES

### Example 1

Gene Synthesis and Expression Vector Construction

Fusion polypeptide-1 DNA was synthesized by custom DNA synthesis and inserted into the multicloning site of pcDNA-3 vector with TOPO cloning kit (Life Technologies). After introduction into E. coli, colonies were picked, and the plasmid DNA was prepared and sequenced. One of the clones that contained the correct sequence was amplified, and the plasmid DNA was isolated using Qiagen Megaprep kit.

Generation of Chinese Hamster Ovary Cell Lines Stably Expressing Fusion Polypeptide-1

The CHO cells were routinely maintained at 37° C. in DMED/F12 medium (Life Technologies, CA) supplemented with 5% (v/v) fetal bovine serum, 2 mM L-glutamax (Life Technologies) in a 5% CO₂ humidified incubator. Prior to transfection, CHO cells (0.5×10⁶ cells) were seeded in a 6-well culture plate containing 2 mL of the complete DMEM/F12 medium. When reached to approximate 80% confluent, the cells were transfected with 2 μg of fusion polypeptide-1 expression plasmid using Lipofectamine™ 2000 reagents (Life Technologies) following manufacturer's instruction.

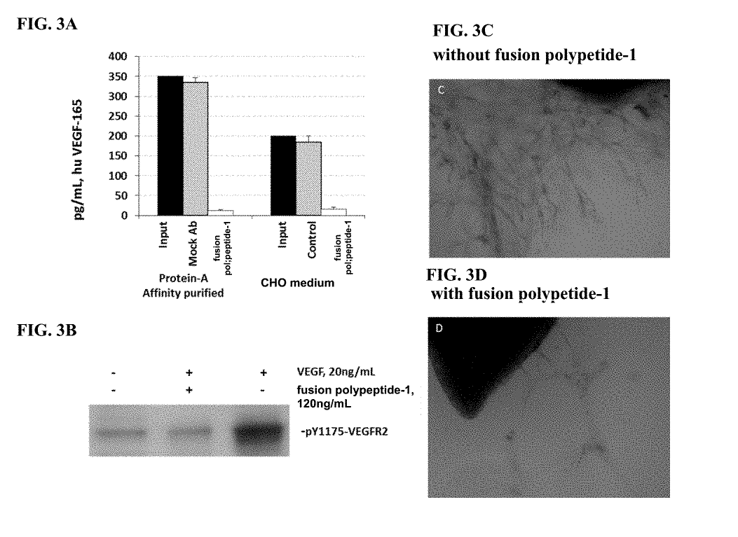

The cells were maintained in transfection for overnight and then washed, trypsinized, and replated into 3 new 10-cm plates containing 1/10, 3/10, and 6/10 of total cell numbers, accordingly. The cells were cultured in complete DMEM/F12 medium containing 600 μg/ml of G418. The selection medium was replenished every 3-4 days until discrete foci of G418-resistant cells were evident after 10-14 days of selection. When the individual colony was clearly formed, 60 individual clones were isolated and expanded in a 48-well plate in the presence of G418. When the cells reached confluence, the culture was gradually adapted into serum-free CHO medium (Life Technologies) by addition of 50% CHO medium for 2 days, and then in 100% of CHO medium for 3-5 days. Expression of fusion polypeptide-1, which comprises an Fc domain, was checked in each clone by dot-blot analysis of culture medium using a HRP-conjugated donkey anti-human Fc antibody (1:10,000 dilution, Jackson ImmunoResearch Laboratories, PA); and revealed with ECL Western blotting substrate (Thermo Scientific, Waltham, Mass.). There were 40 clones showing expression of fusion polypeptide-1 as assayed by dot-blotting on the culture medium, among which 16 clones exhibited strong reactivity (FIG. 3A). The culture medium from randomly selected positive clones after second round selection was further analyzed by Western blotting for protein size and integrity (FIG. 3B). The fusion polypeptide-1 was shown as a 62 kDa single band on a reducing SDS-PAGE gel, which was slightly larger than the theoretically calculated molecular weight of 52 kDa, presumably due to post-translational protein modification. 12 clones with strong reactivity were selected for further cloning by limited dilution in 96-well plates or by single colony picking methods. Still positive clones by dot-blotting were used for initial analysis of VEGF neutralization activity of the fusion polypeptide. The positive clones were then subjected to a second round of cloning by limited dilution in a serial of 96-well plates. The clones, confirmed by dot-blots, were used for protein production.

Large-Scale Cell Culture

Production of fusion polypeptide-1-Fc fusion polypeptide was performed using standard laboratory CHO cell culture system. The fusion polypeptide-1 positive CHO cells were cultured at 37° C., 8% CO₂, in SF-CHO medium with 2 mM L-Glutamax at a seeding density of 0.5×10⁶ cells/mL in a shake flask at 80 rpm. The culture medium was added every 2-3 days to keep a cell density of 0.5 to 1.5×10⁶ cells/mL. When culture medium was added to 300 mL with cell density of 1×10⁶ cells/mL, the L-glutamax was increased to 8 mM. Cell viability was assessed by the trypan blue exclusion test (Life Technologies). When the viable cells dropped to −80%, the cells were separated by centrifugation at 1,000×g for 10 min, and the supernatant was further cleared by high speed centrifugation of 10,000×g for 1 hr at 4° C. The supernatant was used for fusion polypeptide-1 purification.

Purification of Fusion Polypeptide-1

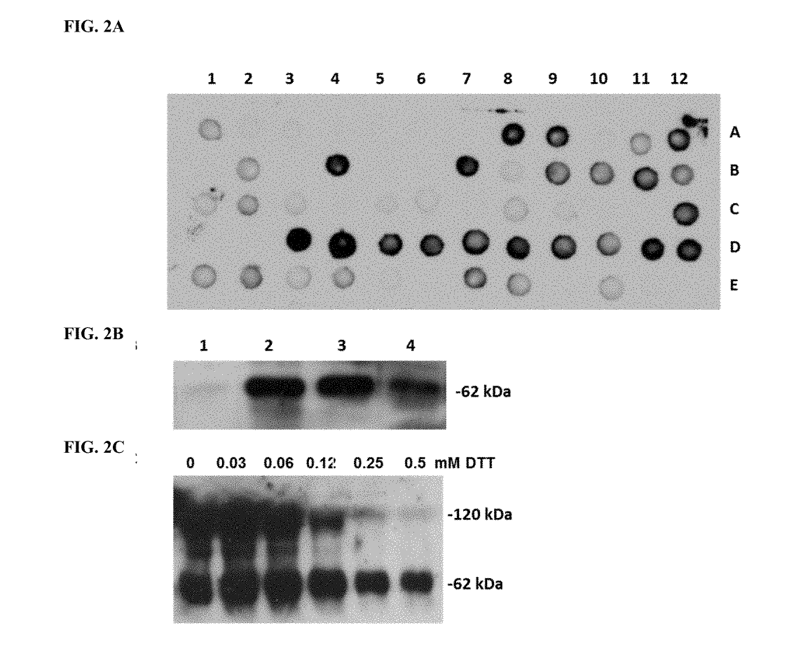

The secreted fusion polypeptide-1 from CHO cells was purified by affinity chromatography of protein G Sepharose™ 4 fast flow (GE Healthcare, Sweden). Cell-free culture supernatant containing fusion polypeptide-1 was adjusted to pH 8.5 with 1 M Tris-HCl, pH 9.0, and loaded onto an Econo column at a flow rate of 50 mL/h. After extensive washing of the column with 0.025 M Tris-HCl, 0.15 M NaCl, pH 8.5, fusion polypeptide-1 was eluted with 0.1 M glycine-HCl, pH 3.0 at a flow-rate adjusted to 30 mL/h. The eluted fraction in a collection of 1-mL serial was rapidly neutralized with 1 M Tris-HCl, pH 9.5. One and five μL from each collected fraction were analyzed by dot-blotting for the presence of fusion polypeptide-1 (see FIG. 2A). The positive fractions were pooled and dialyzed against 1 L of 1×PBS per each for 3 changes. The concentration of purified protein was determined by ELISA.

Detection of Fusion Polypeptide-1 Expression by Dot-Blotting and Western Blotting

For dot-blotting of the fusion polypeptide-1 expression in SF-CHO cell culture medium, 10 μL of culture medium were spotted onto Immobilon-P transfer membrane (Millipore) and allowed to air-dry for 30-60 min. The membrane was blocked in 5% dry milk in Tris-buffered saline containing 0.1% Tween 20 (TBST) for 1 h, and incubated with HRP-conjugated donkey anti-human IgG Fcγ fragment (1:10,000 dilution, Jackson ImmunoReseach) for 1 h. The membrane was then washed three times in TBST prior to detection with ECL Western blotting substrates (Thermo Scientific). For Western analysis of fusion polypeptide-1 expression, the purified protein was diluted in 1× NuPAGE LDS sample buffer (Life Technologies) in the presence of serial concentration of DTT (see FIG. 2C), and separated by SDS-PAGE electrophoresis, transferred onto an Immobilon-P membrane and immunoblotted for human IgGy Fc fragment, as described above (see FIG. 2B).

Quantification of Fusion Polypeptide-1 by ELISA

The fusion polypeptide-1 was quantified with a sandwich enzyme-linked immunosorbent assay (ELISA), based on the capture of the fusion polypeptide-1 to the solid phase of an ELISA plate by goat anti-human IgG Fcγ (Jackson ImmunoResearch Laboratories), and detection of the captured Fc fusion polypeptide via horseradish peroxidase (HRP)-conjugated donkey anti-human IgG Fcγ fragment (Jackson ImmunoResearch laboratories). Briefly, ELISA plates (Nunc Maxisorp™ ELISA plate) were coated with goat anti-human IgG, Fcγ at 1 μg/ml in sodium carbonate, pH 9.5 for overnight at 4° C. The wells were washed three times with PBS containing 0.05% Tween 20 (PBST), and then blocked with PBS containing 1% BSA for 1 h at room temperature. Purified fusion polypeptide-1 was serially diluted with PBS and added to the wells for 2 h at room temperature. After five washes with PBST, 100 μL of HRP-conjugated donkey anti-human IgG, Fcγ (1:10,000 dilution) was added for 1 h. After a final 5 washes in PBST, the colorimetric endpoint was generated using 3,3′,5,5′-Tetramethylbenzidine (TMB)-ELISA substrate (Sigma-Aldrich). The enzyme reaction was stopped by adding 100 μL of 2N sulphuric acid to each well. The optical density was read with an ELISA plate reader (Molecular Devices) at 450 nm. ELISA SoftMax® Pro-5 software was utilized for processing the standard curve and for calculation of the amount of fusion polypeptide-1 in the samples.

In Vitro Depletion of Recombinant Human VEGF-165 by Fusion Polypeptide-1

To determine the VEGF binding activity of the expressed fusion polypeptide-1 fusion polypeptide, we performed in vitro VEGF-165 depletion assay using either the fusion polypeptide-1 expressing CHO cell culture medium or the protein-A agarose bead concentrated fusion polypeptide from the cultured media. 1.5 mL of the fusion polypeptide-1 expression CHO cultured media or the purified fusion polypeptide-1 at a serial concentration were incubated with 200 pM and 350 pM respectively of human VEGF165 at 4° C. for 2 hr with rotating, followed by addition of 20 μL of 50% protein A agarose slurry in 20 mM Tris-HCl, pH8.0. The incubation continued for one more hour before spinning down the bound fusion polypeptide-1-VEGF complex by centrifugation at 12,000×g for 5 min. The supernatant were assayed for the concentration of unbound VEGF by VEGF ELISA kit (Cat# KHG0112, Life Technologies). Both the protein-A bead concentrated protein and the direct culture medium were demonstrated to completely pull-down the input VEGF-165 at concentration of 350 pg/mL and 200 pg/mL, respectively (FIG. 3A), indicating that the positive clones, as selected by dot-blotting using anti-human IgG-Fcγ antibody, can produce a fusion polypeptide possessing the ligand binding capacity.

Neutralization of VEGF Activity by Fusion Polypeptide-1 in Ligand-Induced Receptor Autophosphorylation

To determine the ligand neutralization function for fusion polypeptide-1 protein, a VEGF receptor signal transduction assay was adapted in which the activation of VEGFR2 by VEGF was denoted by phosphorylation at tyrosine 1175 [5]. Functional blocking of VEGF-induced receptor phosphorylation was examined in human Umbilical Vein Endothelial Cell (Huvec). The Huvec cell obtained from ATCC was cultured in F-12K medium supplemented with 0.1 mg/mL of heparin, 0.03 mg/mL endothelial cell growth supplement (ECGS). When cells grew to 90% confluent, the culture media were changed to F-12K medium containing 0.5% FBS but without ECGS and cultured for overnight. Then the cells were challenged for 15 min with vehicle, 20 ng/ml of human VEGF-165 alone or the VEGF-165 that had been pre-incubated at RT for 60 min with 2-fold molar excess of fusion polypeptide-1 in F-12K medium. Cells were then lysed, and immunoblotted with primary antibody specific for phospho-VEGF receptor 2 (Tyr1175) (Cell signaling Technology). Results showed phosphorylation at VEGFR2 tyrosine residue 1175 was rapidly activated by VEGF 165 in Huvec cell, but this activation was blocked by preincubation of the ligand with fusion polypeptide-1 (FIG. 3B), indicating a complete trap of the ligand, VEGF165, by fusion polypeptide-1.

Neutralization of VEGF Activity by Fusion Polypeptide-1—Inhibition of New Blood Vessel Growth in Choroid Sprouting In Vitro

Neutralization of VEGF by fusion polypeptide-1 prompted us to further test in vitro inhibition of microvessel sprouting. Choroid/sclera were isolated from enucleated eyes and cut into 2×1 mm squares, and placed in 24-well plate coated with 30 μL of growth factor-reduced Matrigel™ (BD Biosciences, CA). The Matrigel with choroid was incubated at 37° C. for 10 min, prior to addition of 500 μL of medium into each well for matrigel to solidify. The choroid was cultured in CSC complete medium (Cell Systems) supplemented with EGM-2 medium (Lonza) containing VEGF and 2% FBS, at 37° C., 5% CO₂ for 48 h, in the presence or absence of 100 ng/mL of fusion polypeptide-1. The images were captured on a Zeiss Axio microscope. Comparing with the control treatment, fusion polypeptide-1 significantly inhibited the growth of blood vessels from choroidal/sclera explants (FIGS. 2C and 2D). The inhibition of vessel growth by fusion polypeptide-1 was estimated to be 90% at the concentration of 300 ng/ml.

### Example 2

Compare In Vitro VEGF Binding by Fusion Polypeptide-1 vs Commercial Aflibercept and Conbercept

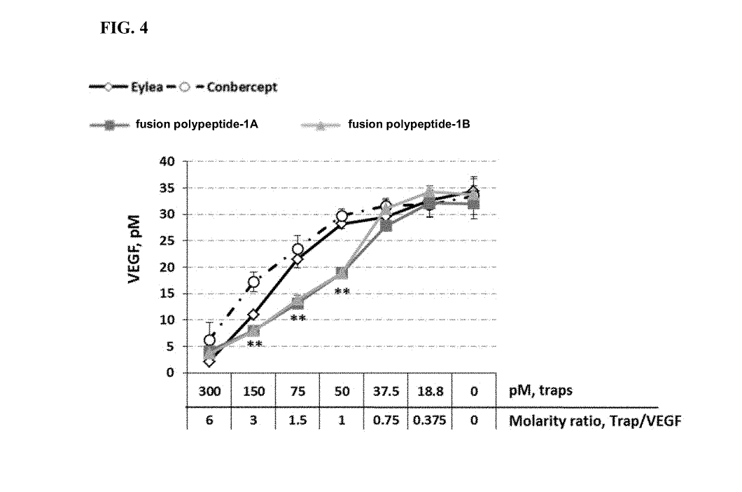

Aflibercept (commercial name, EYLEA) is an Fc-fusion polypeptide carrying extracellular domains of VEGF receptors, being used as decoy receptor to neutralize VEGF for treating the patients with Neovascular (Wet) Age-related Macular Degeneration (AMD), Macular Edema following Retinal Vein Occlusion (RVO), Diabetic Macular Edema (DME) and Diabetic Retinopathy (DR). Conbercept is another Fc-tagged VEGF decoy receptor developed similar to Aflibercept, which is sold in Chinese market. To compare VEGF binding capacity, we performed in vitro VEGF depletion assay by fusion polypeptide-1, Aflibercept and Conbercept. 50 pM (100 pg in 100 μL) of recombinant human VEGF 165 was incubated with each of three molecules at seven different amounts of 300, 150, 75, 50, 37.5, 18.8, and 0 pM, which represent trap receptor-to-VEGF molarity ratio of 6, 3, 1.5, 1, 0.75, 0.375, and 0, accordingly. At high concentration of 300 pM (Trap/VEGF molar ratio of 6:1), all three trap receptors were able to completely deplete the VEGF added, but gradually lose their neutralization capacity with decreasing amount of trap receptors. Interestingly, at 150, 75, and 50 pM of trap receptors per reaction, representing the receptor to ligand molar ratio of 3:1, 1.5:1, and 1:1 accordingly, both Aflibercept and Conbercept pulled down significant less amount of VEGF than either one of two fusion polypeptide-1 preparations from two independent clones (FIG. 4). At 1.5:1 and 1:1 molar ratio conditions, either Aflibercept or Conbercept could only pull down approximately one half amount of the VEGF that was pulled down by fusion polypeptide-1 of either preparations (FIG. 4), suggesting that fusion polypeptide-1 possesses higher VEGF depleting activity. The scheme to construct fusion polypeptide-1 has overcome the shortcomings of VEGFR1 domain 2 Fc fusion polypeptides in literature; the latter only yields 1/60 of VEGF binding affinity of VEGFR1.

### Example 3

Pharmacokinetics Study of Fusion Polypeptide-1 Fusion Polypeptide

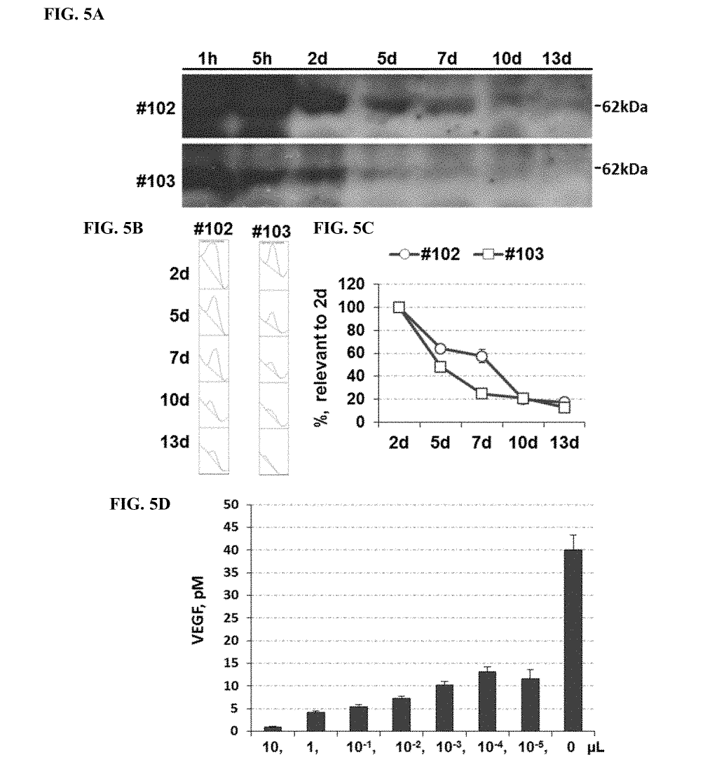

To assess in vivo pharmacokinetic of the fusion polypeptide-1, we injected a low dose of the protein-G agarose purified protein at 0.4 mg per kg of body weight via tail vein into either C57BL6 or Balb/C wild type mice. Blood was collected at 1 and 5 hr post injection at the injection day, and then at day-2 (D2), -5, -7, -10 and -13 via retro-orbital venous plexus (0.1 mL of blood per mouse using a Fishbrand™ Micro Blood collecting tube (Fisher Scientific) or a Microvette® capillary blood collection tube (Sarstedt) under anesthesia. The retention of the injected Fc-tagged fusion polypeptides in serum was analyzed by Western blotting using anti-human IgG-Fcγ antibody. Although the blood contents of Fc-fusion polypeptide decreased gradually within first 7 days after i.v. injection, the constant existence of the Fc-reactive protein was clearly evident by day-10 and -13 post-injection (FIGS. 5A, 5B, and 5C), indicating a stable and long lasting retention of the fusion polypeptide-1 protein in the body fluid. The results from two separate animals (#102 and #103) are shown in the figure.

To further evaluate the neutralization activity of the plasma fusion polypeptide-1 after a long period in circulation, we randomly selected one serum sample isolated from injected animal on day 7 post-injection for in vitro VEGF depletion assay. Fifty pM (100 pg/0.1 mL) of free VEGF 165 were used in each of the binding reaction containing 20 μL serum at a serial of 1:10 dilutions. It was clearly evident that, after 7 days in circulation, the fusion polypeptide-1 still possessed high VEGF binding activity (FIG. 5D). These results demonstrate that fusion polypeptide-1 protein not only survives for a long period in circulation, but also retain its ligand binding activity.

Pharmacodynamic Comparison of Fusion Polypeptide-1 with Ranibizumab (LUCENTIS) and Aflibercept (EYLEA)

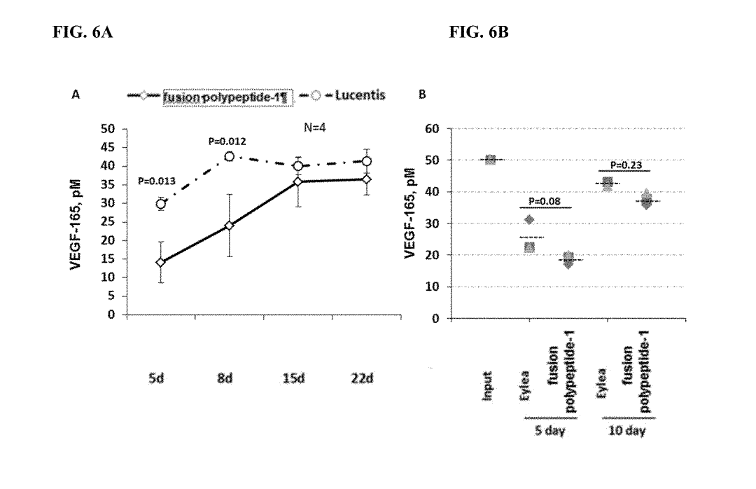

Given that the fusion polypeptide-1 sustained its activity for a long period in circulation, we then performed PD comparison studies of fusion polypeptide-1 with those of either ranibizumab or Aflibercept. In the fusion polypeptide-1 and ranibizumab comparison assay, both fusion polypeptide-1 and ranibizumab were intravenously injected with 0.4 mg per kg of body weight, and serum was prepared at days-5, -8, -15, and -22 post-injection. 20 μL of serum from each sample was used to neutralize 100 ng of recombinant human VEGF 165 in 0.1 mL of assay solution (approximately equal to 50 pM). While all samples from ranibizumab injected mice at 8 days or longer after injection lost most of their VEGF neutralization activity, those from the fusion polypeptide-1 injected mice retained most of the neutralization activity at day-8, and the sera from two of four fusion polypeptide-1 injected mice retained the neutralization activity up to 22 days of post-injection (FIG. 6A), suggesting that the fusion polypeptide-1 has a longer retention and functional activity in vivo, as compared to the antibody-based therapeutics.

In a similar experimental design, we compared PD of fusion polypeptide-1 with Aflibercept, another Fc Fusion based decoy trap receptor that is widely used in clinical medication of the neovascularization-related ophthalmologic diseases. Since both fusion polypeptide-1 and Aflibercept share similar molecular design and molecular weight, we injected equal amount of protein per animal. The serum was prepared after 5 and 10 days, and used in VEGF neutralization assay, as described above. Both fusion polypeptide-1 and Aflibercept were able to neutralize VEGF (FIG. 6B). fusion polypeptide-1 injected serum exhibited a trend of stronger VEGF depleting activity.

### Example 4

Inhibition of Neovascularization by Fusion Polypeptide-1 in the Mouse Models of Laser Injury Induced Choroid Neovascularization (CNV) and Retinopathy of Prematurity (ROP)

VEGF plays crucial role in promoting neovascularization in retina, which is hallmark for a variety of vision threatening ocular diseases, such as wet age-related macular degeneration (AMD), macular edema following retinal vein occlusion (RVO), diabetic macular edema (DME), diabetic retinopathy (DR), and retinopathy of prematurity (ROP). To determine the anti-CNV activity of fusion polypeptide-1 in vivo, we adopted a laser-induced CNV model [1,3], and evaluated the development of new blood vessel complex in the retinas of the mice that had been treated with laser burn for 7 days in the presence or absence of fusion polypeptide-1.

Laser Injury Induced Choroid Neovascularization (CNV)

The laser-induced neovascularization was performed on mice at ages between 6-10 weeks [1,2]. The pupils were dilated with a single drop mixture of 0.06% tropicamide and 0.3% phenylephrine hydrochloride. Two minutes later, the mice were anesthetized with intraperitoneal injection of Ketamine-Xylazine solution (100 mg/kg and 10 mg/kg body weight for Ketamine and Xylazine, respectively). Diode laser burns applied with a Novus Spectra ophthalmic laser (Lumenis, Inc., Santa Clara, Calif.) mounted on a slit lamp (Model SL-M; Zeiss, Inc., Tokyo, Japan), and generated four lesions symmetrically surrounding the optic nerve of each eye. A coverslip coated with 2.5% hypromellose ophthalmic demulcent solution was held on the mouse cornea, which served to subtract the optics of the cornea and lens for optimal view of the retina and spots of the laser lesions [1,3]. The laser variables were set as 50 μm in spot size, 0.05 seconds duration, and 250 mW in power. The power used was assessed by the ability to produce a blister indicating rupture of the Bruch's membrane. 2 μL of 2 μg of fusion polypeptide-1 was intravitreally injected immediately after the laser injury. Laser spots were evaluated by isolectin GS-IB₄ staining for the presence of CNV at day 7 after laser treatment

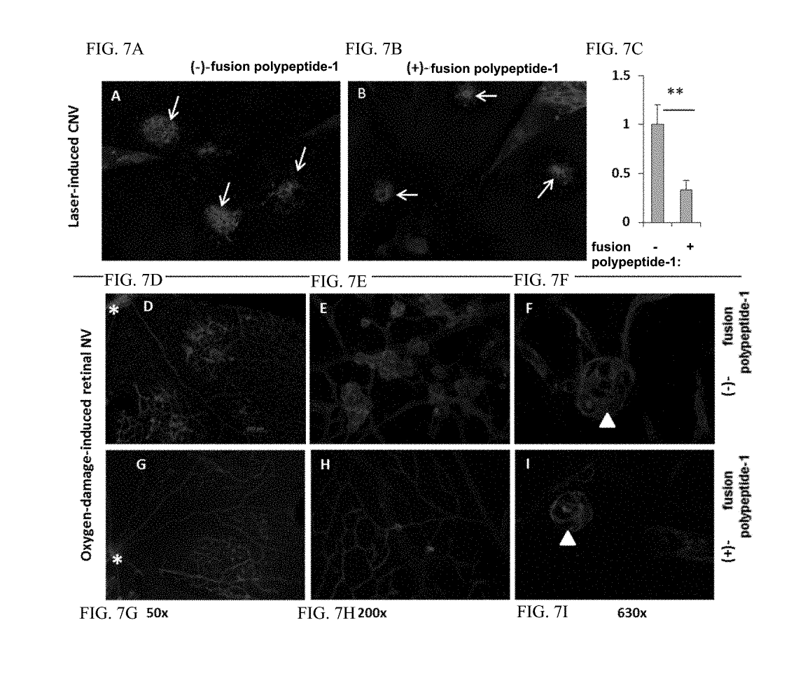

For laser induced CNV, the laser treated eyes were enucleated and fixed at 4° C. with ice-cold 4% paraformaldehyde in PBS for overnight. The anterior segment, lens, and retina were removed, and the remaining eye cups with attached RPE layer were washed with PBS, and proceeded with Isolectin-GS-IB₄ staining. The eye cup was permeabilized by incubation in 0.5% Triton X-100 at 4° C. for overnight. After wash with PBS, the tissue was incubated in a solution of 10 μg/mL of Alexa Fluor 568-conjugated isolectin-GS-IB₄ (Life Technologies) by rocking at room temperature for overnight. After incubation, the stained eye cups were mounted andexamined with a fluorescence microscope. Results showed that CNV formation in the control mouse retinas (FIG. 7A), but this was significantly inhibited by injection with fusion polypeptide-1 (FIGS. 7B and C).

Oxygen-Induced Retinopathy (OIR)

Inhibition of neovascularization by fusion polypeptide-1 was further examined on a mouse model of retinopathy of prematurity, in which new-born pups at postnatal day 7 were placed in a hyperoxic environment for 5 days to cause blood vessels constriction and obliteration of the central retinal vessels. Upon return to normoxia at p12, the central retinal area became hypoxic leading to development of pathological NV, during which time, re-vascularization of the normal plexuses and the formation of pathological, pre-laminar neovascular tufts required the presence of VEGF. To induce neovascularization in new born babies, neonates at postnatal day 7 (P7) and mother were exposed to 75% oxygen in a BioSpherix ProOx Model 110 unit, lasting until P12 when the maximum Vaso-obliteration was induced [4]. Animals were returned to room air for a four hour recovery period before i.p. injection with 2 μg of purified fusion polypeptide-1 in 20 μL saline. When maximal neovascularization occurred five days later at P17, the neonates were sacrificed and the eyes were enucleated under deep anesthesia, and fixed in 4% PFA for 1 h at room temperature. The retina was removed from eye ball and permeabilized by incubation in 0.5% Triton X-100 at 4° C. for overnight. After wash with PBS, the retinas were incubated in a solution of 10 μg/mL of Alexa Fluor 568-conjugated isolectin-GS-IB₄ (Life Technologies) by rocking at room temperature for overnight. After incubation, the retinas with radial cuts were flat mounted for fluorescence microscopy right after the staining.

Five days after switching to normoxia condition, neovascularization was characterized by fluorescently labeled isolectin IB4 staining in the retina, and massive newly formed vascular complex was induced by p17 in the absence of fusion polypeptide-1 (FIG. 7D-F). Such pathological NV development and the size of the induced NV were significantly inhibited by fusion polypeptide-1 treatment (FIG. 7G-I). These results suggest that fusion polypeptide-1 protein has high potential to be developed into a potent therapeutic drug for treating NV related ophthalmologic diseases.

Statistical Analysis

Data was presented as means±standard deviation (SD). The statistical significance and P values were calculated by ANOVA multiple comparison, Tukey's HSD analysis for multiple group comparison, and by one way ANOVA for two group comparison.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Sequence Listing:

VEGFR1 (FLT1) immunoglobulin-like-type 3 domain SEQ ID NO: 1
DVQISTPRPVKLLRGHTLVLNCTATTPLNTRVQMTWSYPDKNKRASVRRRIDQSNS
HANIFYSVLTIDKMQNKDKGLYTCRVRSGPSFKSVNTSVH
VEGFR2 (KDR) immunoglobulin-like-type 3 domain SEQ ID NO: 2
DVVLSPSHGIELSVGEKLVLNCTARTELNVGIDFNWEYPSSKHQHK
KLVNRDLKTQSGSEMKKFLSTLTIDGVTRSDQGLYTCAASSGLMTKKNST
VEGFR3 (FLT4) immunoglobulin-like-type 3 domain SEQ ID NO: 3
PFLVHITGNELYDIQLLPRKSLELLVGEKLVLNCTVWAEFNSGVTFDWDYPGKQAER
GKWVPERRSQQTHTELSSILTIHNVSQHDLGSYVCKANNGIQRFRESTEVI
VEGFR1 (FLT1) immunoglobulin-like-type 2 domain SEQ ID NO: 4
GRELVIPCRVTSPNITVTLKKFPLDTLIPDGKRIIWDSRKGFIISNATYKEIGLLTCEAT
VNGH
VEGFR2 (KDR) immunoglobulin-like-type 2 domain SEQ ID NO: 5
NKNKTVVIPCLGSISNLNVSLCARYPEKRF VPDGNRISWDSKKGFTIP SYMISYAGMV
FCEAKINDE
VEGFR3 (FLT4) immunoglobulin-like-type 2 domain SEQ ID NO: 6
KDAMWVPCLVSIPGLNVTLRSQSSVLWPDGQEVVWDDRRGMLVSTPLLHDALYLQ
CETTWGDQ
Polynucleotide that encodes fusion polypeptide-1 SEQ ID NO: 7
ACG CGT GCC ACC ATG GTC AGC TAC TGG GAC ACC GGG GTC CTG CTG TGC
GCG CTG CTC AGC TGT CTG CTT CTC ACA GGA TCT AGT TCC GGA AGT GAT ACC
GGT AGA CCT TTC GTA GAG ATG TAC AGT GAA ATC CCC GAA ATT ATA CAC ATG
ACT GAA GGA AGG GAG CTC GTC ATT CCC TGC CGG GTT ACG TCA CCT AAC ATC
ACT GTT ACT TTA AAA AAG TTT CCA CTT GAC ACT TTG ATC CCT GAT GGA AAA
CGC ATA ATC TGG GAC AGT AGA AAG GGC TTC ATC ATA TCA AAT GCA ACG TAC
AAA GAA ATA GGG CTT CTG ACC TGT GAA GCA ACA GTC AAT GGG CAT TTG TAT
AAG ACA AAC TAT CTC ACA CAT CGA CAA ACC AAT ACA ATC ATA GAT GGT GGA
GGC GGA TCG GGA GGC GGT GGG TCC CCG TCA GTC TTC CTC TTC CCC CCA AAA
CCC AAG GAC ACC CTC ATG ATC TCC CGG ACC CCT GAG GTC ACA TGC GTG GTG
GTG GAC GTG AGC CAC GAA GAC CCT GAG GTC AAG TTC AAC TGG TAC GTG GAC
GGC GTG GAG GTG CAT AAT GCC AAG ACA AAG CCG CGG GAG GAG CAG TAC AAC
AGC ACG TAC CGT GTG GTC AGC GTC CTC ACC GTC CTG CAC CAG GAC TGG CTG
AAT GGC AAG GAG TAC AAG TGC AAG GTC TCC AAC AAA GCC CTC CCA GCC CCC
ATC GAG AAA ACC ATC TCC AAA GCC AAA GGG CAG CCC CGA GAA CCA CAG GTG
TAC ACC CTG CCC CCA TCC CGG GAT GAG CTG ACC AAG AAC CAG GTC AGC CTG
ACC TGC CTG GTC AAA GGC TTC TAT CCC AGC GAC ATC GCC GTG GAG TGG GAG
AGC AAT GGG CAG CCG GAG AAC AAC TAC AAG ACC ACG CCT CCC GTG CTG GAC
TCC GAC GGC TCC TTC TTC CTC TAC AGC AAG CTC ACC GTG GAC AAG AGC AGG
TGG CAG CAG GGG AAC GTC TTC TCA TGC TCC GTG ATG CAT GAG GCT CTG CAC
AAC CAC TAC ACG CAG AAG AGC CTC TCC CTG TCT CCG GGT AAA ACT CAC ACA
TGC CCA CCG TGC CCA GCA CCT GAA CTC CTG GGG GGA GGA TCC CGG CCA TTT
GTT GAA ATG TAT TCA GAG ATT CCT GAG ATC ATT CAT ATG ACA GAG GGC CGA
GAA TTG GTA ATA CCA TGC AGA GTC ACC AGT CCC AAT ATA ACA GTC ACC CTG
AAG AAA TTC CCT CTC GAT ACG CTC ATT CCA GAT GGC AAA AGG ATC ATA TGG
GAT TCA CGC AAG GGA TTT ATA ATC AGT AAC GCA ACC TAT AAA GAG ATC GGA
TTG CTT ACT TGT GAG GCA ACG GTA AAC GGT CAC CTT TAC AAG ACG AAT TAC
TTG ACT CAC AGG CAA ACG AAC ACT ATA ATT GAC TGA GGA TCC AAA TGA
Flt1 immunoglobulin-like-type 3 domain Basic Region SEQ ID NO: 8
KNKRASVRR
Aflibercept SEQ ID NO: 9
SDTGRPFVEMYSEIPEIIHMTEGRELVIPCRVTSPNITVTLKKFPLDTLIPDGKRIIWDSRKGFII
SNATYKEIGLLTCEATVNGHLYKTNYLTHRQTNTIIDVVLSPSHGIELSVGEKLVLNCTARTE
LNVGIDENWEYPSSKHQHKKLVNRDLKTQSGSEMKKFLSTLTIDGVTRSDQGLYTCAASSG
LMTKKNSTFVRVHEKDKTHTCPPCPAPELLGGPSVFLEPPKPKDTLMISRTPEVTCVVVDVS
HEDPEVKFNWYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG

1. A fusion polypeptide comprising two or more VEGF receptor immunoglobulin-like-type 2 domains fused with a dimerization polypeptide, wherein at least one of said two or more VEGF receptor immunoglobulin-like-type 2 domains is fused to an N-terminus of said dimerization polypeptide and at least another of said two or more VEGF receptor immunoglobulin-like-type 2 domains is fused to a C-terminus of said dimerization polypeptide.

2. (canceled)

3. The fusion polypeptide of claim 1, wherein said dimerization polypeptide is a fragment crystallizable (Fc) domain.

4. The fusion polypeptide of claim 1, wherein said dimerization domain comprises a first cysteine residue capable of forming a disulfide bond to a second cysteine residue.

5. The fusion polypeptide of claim 1, further comprising at least one hinge region between said dimerization polypeptide and said two or more VEGF receptor immunoglobulin-like-type 2 domains.

6. The fusion polypeptide of claim 1, wherein said two or more VEGF receptor immunoglobulin-like-type 2 domains:
are each at least 80% identical to a human VEGF receptor immunoglobulin-like-type 2 domain selected from the group consisting of SEQ ID NOs 4-6;
are each at least 90% identical to a human VEGF receptor immunoglobulin-like-type 2 domain selected from the group consisting of SEQ ID NOs 4-6;
are each at least 95% identical to a human VEGF receptor immunoglobulin-like-type 2 domain selected from the group consisting of SEQ ID NOs 4-6;
are each a human VEGF receptor immunoglobulin-like-type 2 domain selected from the group consisting of SEQ ID NOs 4-6;
are each SEQ ID NO: 4;
are each SEQ ID NO: 5; or
are each SEQ ID NO: 6.

7. (canceled)

8. (canceled)

9. (canceled)

10. (canceled)

11. (canceled)

12. (canceled)

13. The fusion polypeptide of claim 1, wherein said two or more VEGF receptor immunoglobulin-like-type 2 domains are not identical.

14. The fusion polypeptide of claim 13, wherein said two or more VEGF receptor immunoglobulin-like-type 2 domains are at least two distinct human VEGF receptor immunoglobulin-like-type 2 domains.

15. The fusion polypeptide of claim 14, wherein said at least two distinct human VEGF receptor immunoglobulin-like-type 2 domains are selected from the group consisting of SEQ ID NOs: 4-6.

16. The fusion polypeptide of claim 1, wherein said fusion polypeptide substantially lacks a VEGF receptor immunoglobulin-like-type 3 domain selected from the group consisting of SEQ ID NOs 1-3.

17. The fusion polypeptide of claim 1, wherein said fusion polypeptide comprises no more than 60 amino acids of a VEGF receptor immunoglobulin-like-type 3 domain selected from the group consisting of SEQ ID NOs 1-3.

18. The fusion polypeptide of claim 16, wherein said VEGF receptor immunoglobulin-like-type 3 domain is:
at least 80% identical to a human VEGF receptor immunoglobulin-like-type 3 domain selected from the group consisting of SEQ ID NOs 1-3;
at least 90% identical to a human VEGF receptor immunoglobulin-like-type 3 domain selected from the group consisting of SEQ ID NOs 1-3;
at least 95% identical to a human VEGF receptor immunoglobulin-like-type 3 domain selected from the group consisting of SEQ ID NOs 1-3;
SEQ ID NO: 1;
SEQ ID NO: 2; or
SEQ ID NO: 3.

19. (canceled)

20. (canceled)

21. (canceled)

22. (canceled)

23. (canceled)

24. The fusion polypeptide of claim 1, wherein said fusion polypeptide does not comprise SEQ ID NO: 8.

25. The fusion polypeptide of claim 1, wherein a homodimer of said fusion polypeptide exhibits high-affinity binding to VEGF.

26. The fusion polypeptide of claim 25, wherein said homodimer binds to VEGF with a higher affinity than the polypeptide shown in SEQ ID NO: 9.

27. The fusion polypeptide of claim 25, wherein said homodimer binds to VEGF with a higher affinity than a human VEGF receptor.

28. (canceled)

29. An isolated polynucleotide molecule encoding said fusion polypeptide of claim 1.

30. The isolated polynucleotide molecule of claim 29, wherein said polynucleotide comprises SEQ ID NO: 7.

31. (canceled)

32. (canceled)

33. (canceled)

34. (canceled)

35. (canceled)

36. (canceled)

37. A method for inhibiting angiogenesis in a subject in need thereof comprising:
administering to said subject in need thereof a therapeutically effective amount of a homodimer of said fusion polypeptide of claim 1.

38. The method of claim 37, wherein said administering is effected by a local administration or a systemic administration to said subject.

39. The method of claim 38, wherein said administration is to an eye of said subject.

40. The method of claim 39, wherein said administration is to a tumor tissue of said subject.

41. The method of claim 39, wherein said administration is intravenous injection, intraperitoneal injection, or intraperitoneal injection.

42. (canceled)

43. (canceled)

44. The method of claim 37, wherein said angiogenesis is a manifestation of a condition selected from the group consisting of age-related macular degeneration, diabetic retinopathy, choroidal neovascularization, cystoid macular edema, diabetic macular edema, retinal vascular occlusion, corneal neovascularization, corneal transplantation, neovascular glaucoma, pterygium chronic conjunctivitis, angiogenesis related therapy failure such as laser coagulation, and surgical retinal transplantation.

45. The method of claim 44, wherein said condition is AMD.

46. The method of claim 44, wherein said condition is diabetic retinopathy.

47. The method of claim 44, wherein said administering results in one or more improved symptoms of said condition, wherein said symptoms are selected from the group consisting of a decrease in mean choroidal neovascularization (CNV) leakage, improved mean visual acuity, a reduction in mean foveal retinal thickness, a reduction in mean macular size, and a reduction in mean lesion size.

48. The method of claim 47, wherein said one or more improved symptoms of said condition remains improved for at least 1 month following said administration.

49. The method of claim 47, wherein said homodimer is administered by intravitreal injection at an amount from about 1 mg to about 3 mg

50. The method of claim 49, wherein said homodimer is administered by intravitreal injection of an amount of about 2 mg.

51. The method of claim 37, wherein said angiogenesis is a manifestation of a tumor.

52. (canceled)

53. The method of claim 51, wherein said fusion polypeptide is administered by an intravenous injection comprising an amount of from about 0.1 to about 30 mg/kg, or from about 1 to about 8 mg/kg.


SEQUENCE LISTING (9 sequences)

SEQ ID NO: 1 - protein, 96 aa, Homo sapiens
DVQISTPRPVKLLRGHTLVLNCTATTPLNTRVQMTWSYPDKNKRASVRRRIDQSNSHANIFYSVLTIDKMQNKDKGLYTCRVRSGPSFKSVNTSVH

SEQ ID NO: 2 - protein, 96 aa, Homo sapiens
DVVLSPSHGIELSVGEKLVLNCTARTELNVGIDFNWEYPSSKHQHKKLVNRDLKTQSGSEMKKFLSTLTIDGVTRSDQGLYTCAASSGLMTKKNST

SEQ ID NO: 3 - protein, 108 aa, Homo sapiens
PFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVI

SEQ ID NO: 4 - protein, 64 aa, Unknown
  Description of Unknown VEGFR1 (FLT1) immunoglobulin-like-type 2 domain
GRELVIPCRVTSPNITVTLKKFPLDTLIPDGKRIIWDSRKGFIISNATYKEIGLLTCEATVNGH

SEQ ID NO: 5 - protein, 67 aa, Unknown
  Description of Unknown VEGFR2 (KDR) immunoglobulin-like-type 2 domain
NKNKTVVIPCLGSISNLNVSLCARYPEKRFVPDGNRISWDSKKGFTIPSYMISYAGMVFCEAKINDE

SEQ ID NO: 6 - protein, 63 aa, Unknown
  Description of Unknown VEGFR3 (FLT4) immunoglobulin-like-type 2 domain
KDAMWVPCLVSIPGLNVTLRSQSSVLWPDGQEVVWDDRRGMLVSTPLLHDALYLQCETTWGDQ

SEQ ID NO: 7 - DNA, 1422 bp, Artificial Sequence
  Description of Artificial Sequence Synthetic polynucleotide
acgcgtgccaccatggtcagctactgggacaccggggtcctgctgtgcgcgctgctcagctgtctgcttctcacaggatctagttccggaagtgataccggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatggtggaggcggatcgggaggcggtgggtccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaactcacacatgcccaccgtgcccagcacctgaactcctggggggaggatcccggccatttgttgaaatgtattcagagattcctgagatcattcatatgacagagggccgagaattggtaataccatgcagagtcaccagtcccaatataacagtcaccctgaagaaattccctctcgatacgctcattccagatggcaaaaggatcatatgggattcacgcaagggatttataatcagtaacgcaacctataaagagatcggattgcttacttgtgaggcaacggtaaacggtcacctttacaagacgaattacttgactcacaggcaaacgaacactataattgactgaggatccaaatga

SEQ ID NO: 8 - protein, 9 aa, Homo sapiens
KNKRASVRR

SEQ ID NO: 9 - protein, 431 aa, Artificial Sequence
  Description of Artificial Sequence Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG